SEQUENCE LISTING (56 sequences)

SEQ ID NO: 1 - protein, 165 aa, Homo sapiens
MAPNASCLCVHVRSEEWDLMTFDANPYDSVKKIKEHVRSKTKVPVQDQVLLLGSKILKPRRSLSSYGIDKEKTIHLTLKVVKPSDEELPLFLVESGDEAKRHLLQVRRSSSVAQVKAMIETKTGIIPETQIVTCNGKRLEDGKMMADYGIRKGNLLFLACYCIGG

SEQ ID NO: 2 - DNA, 894 bp, Homo sapiens
  source 1..894: mol_type = genomic DNA
gtctctggtttctggccccttgtctgcagagatggctcccaatgcttcctgcctctgtgtgcatgtccgttccgaggaatgggatttaatgacctttgatgccaacccatatgacagcgtgaaaaaaatcaaagaacatgtccggtctaagaccaaggttcctgtgcaggaccaggttcttttgctgggctccaagatcttaaagccacggagaagcctctcatcttatggcattgacaaagagaagaccatccaccttaccctgaaagtggtgaagcccagtgatgaggagctgcccttgtttcttgtggagtcaggtgatgaggcaaagaggcacctcctccaggtgcgaaggtccagctcagtggcacaagtgaaagcaatgatcgagactaagacgggtataatccctgagacccagattgtgacttgcaatggaaagagactggaagatgggaagatgatggcagattacggcatcagaaagggcaacttactcttcctggcatgttattgtattggagggtgaccaccctgggcatggggtgttggcaggggtcaaaaagcttatttcttttaatctcttactcaacgaacacatcttctgatgatttcccaaaattaatgagaatgagatgagtagagtaagatttgggtgggatgggtaggatgaagtatattgcccaactctatgtttctttgattctaacacaattaattaagtgacatgatttttactaatgtattactgagactagtaaataaatttttaaggcaaaatagagcattcaaagccagcttggaatttaattctgtcttgataccttgttatttatgcaaaaactcctatctcctttcctttatgacaagagagtaagttttaggttgggatcc

SEQ ID NO: 3 - DNA, 20 bp, synthetic construct
  source 1..20: mol_type = other DNA
tggtcctgcacaggaacctt

SEQ ID NO: 4 - DNA, 20 bp, synthetic construct
  source 1..20: mol_type = other DNA
aggcttctccgtggctttaa

SEQ ID NO: 5 - DNA, 20 bp, synthetic construct
  source 1..20: mol_type = other DNA
ggtgcctctttgcctcatca

SEQ ID NO: 6 - DNA, 20 bp, synthetic construct
  source 1..20: mol_type = other DNA
actttgtcattctcagtggt

SEQ ID NO: 7 - DNA, 20 bp, synthetic construct
  source 1..20: mol_type = other DNA
cactttgtcattctcagtgg

SEQ ID NO: 8 - DNA, 20 bp, synthetic construct
  source 1..20: mol_type = other DNA
cttcactttgtcattctcag

SEQ ID NO: 9 - DNA, 20 bp, synthetic construct
  source 1..20: mol_type = other DNA
ttcttcactttgtcattctc

SEQ ID NO: 10 - DNA, 20 bp, synthetic construct
  source 1..20: mol_type = other DNA
tttgagcttctcatcaccgc

SEQ ID NO: 11 - DNA, 20 bp, synthetic construct
  source 1..20: mol_type = other DNA
atagctcagaggaaatcggc

SEQ ID NO: 12 - DNA, 20 bp, synthetic construct
  source 1..20: mol_type = other DNA
ggtaatagctcagaggaaat

SEQ ID NO: 13 - DNA, 20 bp, synthetic construct
  source 1..20: mol_type = other DNA
ggactccaccagaaacaagg

SEQ ID NO: 14 - DNA, 20 bp, synthetic construct
  source 1..20: mol_type = other DNA
ccgttgcaattcacaacctg

SEQ ID NO: 15 - DNA, 20 bp, synthetic construct
  source 1..20: mol_type = other DNA
agccatctctgcagacaagg

SEQ ID NO: 16 - DNA, 20 bp, synthetic construct
  source 1..20: mol_type = other DNA
gagccatctctgcagacaag

SEQ ID NO: 17 - DNA, 20 bp, synthetic construct
  source 1..20: mol_type = other DNA
ggagccatctctgcagacaa

SEQ ID NO: 18 - DNA, 20 bp, synthetic construct
  source 1..20: mol_type = other DNA
ttgggagccatctctgcaga

SEQ ID NO: 19 - DNA, 20 bp, synthetic construct
  source 1..20: mol_type = other DNA
attgggagccatctctgcag

SEQ ID NO: 20 - DNA, 20 bp, synthetic construct
  source 1..20: mol_type = other DNA
cattgggagccatctctgca

SEQ ID NO: 21 - DNA, 20 bp, synthetic construct
  source 1..20: mol_type = other DNA
agcattgggagccatctctg

SEQ ID NO: 22 - DNA, 20 bp, synthetic construct
  source 1..20: mol_type = other DNA
aagcattgggagccatctct

SEQ ID NO: 23 - DNA, 20 bp, synthetic construct
  source 1..20: mol_type = other DNA
gaagcattgggagccatctc

SEQ ID NO: 24 - DNA, 20 bp, synthetic construct
  source 1..20: mol_type = other DNA
ggaagcattgggagccatct

SEQ ID NO: 25 - DNA, 20 bp, synthetic construct
  source 1..20: mol_type = other DNA
aggaagcattgggagccatc

SEQ ID NO: 26 - DNA, 20 bp, synthetic construct
  source 1..20: mol_type = other DNA
caggaagcattgggagccat

SEQ ID NO: 27 - DNA, 20 bp, synthetic construct
  source 1..20: mol_type = other DNA
agaggcaggaagcattggga

SEQ ID NO: 28 - DNA, 20 bp, synthetic construct
  source 1..20: mol_type = other DNA
acagaggcaggaagcattgg

SEQ ID NO: 29 - DNA, 20 bp, synthetic construct
  source 1..20: mol_type = other DNA
cacagaggcaggaagcattg

SEQ ID NO: 30 - DNA, 20 bp, synthetic construct
  source 1..20: mol_type = other DNA
acacagaggcaggaagcatt

SEQ ID NO: 31 - DNA, 20 bp, synthetic construct
  source 1..20: mol_type = other DNA
cacacagaggcaggaagcat

SEQ ID NO: 32 - DNA, 20 bp, synthetic construct
  source 1..20: mol_type = other DNA
atgcacacagaggcaggaag

SEQ ID NO: 33 - DNA, 20 bp, synthetic construct
  source 1..20: mol_type = other DNA
catgcacacagaggcaggaa

SEQ ID NO: 34 - DNA, 20 bp, synthetic construct
  source 1..20: mol_type = other DNA
acatgcacacagaggcagga

SEQ ID NO: 35 - DNA, 20 bp, synthetic construct
  source 1..20: mol_type = other DNA
aacggacatgcacacagagg

SEQ ID NO: 36 - DNA, 20 bp, synthetic construct
  source 1..20: mol_type = other DNA
gaacggacatgcacacagag

SEQ ID NO: 37 - DNA, 20 bp, synthetic construct
  source 1..20: mol_type = other DNA
ggaacggacatgcacacaga

SEQ ID NO: 38 - DNA, 20 bp, synthetic construct
  source 1..20: mol_type = other DNA
tcggaacggacatgcacaca

SEQ ID NO: 39 - DNA, 20 bp, synthetic construct
  source 1..20: mol_type = other DNA
atgccataagatgagaggct

SEQ ID NO: 40 - DNA, 20 bp, synthetic construct
  source 1..20: mol_type = other DNA
aatgccataagatgagaggc

SEQ ID NO: 41 - DNA, 20 bp, synthetic construct
  source 1..20: mol_type = other DNA
caatgccataagatgagagg

SEQ ID NO: 42 - DNA, 20 bp, synthetic construct
  source 1..20: mol_type = other DNA
gtcttctctttgtcaatgcc

SEQ ID NO: 43 - DNA, 20 bp, synthetic construct
  source 1..20: mol_type = other DNA
ggtcttctctttgtcaatgc

SEQ ID NO: 44 - DNA, 20 bp, synthetic construct
  source 1..20: mol_type = other DNA
catgccaggaagagtaagtt

SEQ ID NO: 45 - DNA, 20 bp, synthetic construct
  source 1..20: mol_type = other DNA
acatgccaggaagagtaagt

SEQ ID NO: 46 - DNA, 20 bp, synthetic construct
  source 1..20: mol_type = other DNA
aacatgccaggaagagtaag

SEQ ID NO: 47 - DNA, 20 bp, synthetic construct
  source 1..20: mol_type = other DNA
caataacatgccaggaagag

SEQ ID NO: 48 - DNA, 20 bp, synthetic construct
  source 1..20: mol_type = other DNA
gctggctttgaatgctctat

SEQ ID NO: 49 - DNA, 20 bp, synthetic construct
  source 1..20: mol_type = other DNA
agctggctttgaatgctcta

SEQ ID NO: 50 - DNA, 20 bp, synthetic construct
  source 1..20: mol_type = other DNA
aagctggctttgaatgctct

SEQ ID NO: 51 - DNA, 20 bp, synthetic construct
  source 1..20: mol_type = other DNA
caagctggctttgaatgctc

SEQ ID NO: 52 - DNA, 20 bp, synthetic construct
  source 1..20: mol_type = other DNA
ccaagctggctttgaatgct

SEQ ID NO: 53 - DNA, 20 bp, synthetic construct
  source 1..20: mol_type = other DNA
tccaagctggctttgaatgc

SEQ ID NO: 54 - DNA, 20 bp, synthetic construct
  source 1..20: mol_type = other DNA
ttccaagctggctttgaatg

SEQ ID NO: 55 - protein, 162 aa, Mus musculus
MASVRTCVVRSDQWRLMTFETTENDKVKKINEHIRSQTKVSVQDQILLLDSKILKPHRKLSSYGIDKETTIHLTLKVVKPSDEELPLFLVESKNEGQRHLLRVRRSSSVAQVKEMIESVTSVIPKKQVVNCNGKKLEDGKIMADYNIKSGSLLFLTTHCTGG

SEQ ID NO: 56 - DNA, 2210 bp, Mus musculus
  source 1..2210: mol_type = genomic DNA
agctactcacatataagcagacacaaggactcttttcttattcagcctctgactacagacatggcttctgtccgcacctgtgtaagttggggtttggtaaacgaatgggaaggaaaagtatttccatgtattgggggtacagttccacaacttgggaggggacagtggtctagaaggtgaggcagttgggtgagaaacatttaggagaaaaggcagacattgataactgcacaataggaagagggtactctgaagagaggttggtcatttgacttattaaacttttaaaatattttatttattatatgtgtgcatgtatatggtgggtcagagccagaagataaccttcaggagtcagttctctctcttcaccacataggttctagggatcaagcttaaaggtgaggcttggcagcaagagattttattgactgactgattgattgattgatttacttatttgttatttatttaaacagctgagccatttttgccatccccacttaggagaaatggtaggacactttgcttcatgaggctggcaagatgtgaaattaggaaaataacttacagagttctcagccttaagcgtctctctcttttcaaaggaaaacttcataaaacttgtttttcttgttattggtgacataactaacacattaccttaaacaggttatgctccaatgtaaagaatgtgcaattagggcaatgtccaatacaagacaaaactgaagaagaatatggaatatctaggttgggaaggttccttgaatggagtatgaggaagttttcataaagtttacaggcctgattcttccctaacttaattcttcagtaaatgaagttaaactcagatatagtttttgtcttctgtgaatgggatcctggtctaatttaaaaaggatattatgttctatatcaatggatataaaatacaagagatacacattaacaccattaagcacagcatcaccacaacatggacattaccagaaatggacgggaacattaataagaacgcagcagtgtagagactatgtcttaactagggttctttctgaattagtcacagagggtgagtcatggctgagagcattctgtgaggctgccaaatcttggaaatcctctttgggtttgcttagggatctgtatacaacgtttgaagccctatagtttcacgaagccgatgttctgatttgggttatgatgtaggaatcaaaagttgcaagagagggagagatgcagactgttgagaaggagccagagggccaaccaggagaacagtgaagctgaggagtagggggtagagggtggcaggcgttcttagatgacgctcttggctttctttgcctaggttgtccgttcagaccaatggcggttaatgacctttgagaccactgagaatgacaaagtgaagaagataaatgaacatattaggtcccaaaccaaggtctctgtacaggaccagatccttctgctagactccaaaatcctcaagccccatcgaaaattgtcatcctatgggattgacaaggaaaccactatccaccttaccctgaaggtggtgaagcccagtgatgaagagctgcccttgtttctggtggagtccaaaaacgaggggcaaaggcacctcctccgagttcgaagatccagctcagtggcccaggtgaaagagatgatcgagagtgtgacctctgtgatccctaagaagcaggttgtgaattgcaacggaaagaagctggaagatggaaagatcatggctgactacaacatcaagagtggcagtttgctctttctgacaacacactgcactgggggatgaccacggggatgcggtgatgagaagctcaaagccgatttcctctgagctattaccaatcacatctcttgatgatataaaaaattaacgagaatgaaataaatatttgagggaagtggagtagattaaaagtatttgtcaagtctgtgaatcttgtttctaacatagttcattcgcagggttgttgatgagggcaaaagctgaacctttagtcaggtgtggtggtgcaaacttaattccaggacttgggatgcaggggaaggtggatcaatgaatgattgttgaaccaaattgaattttaattatagagtatagagaaagaatgtgttatggaatatgattgtttaccataactggatcatagtctaataaattatttaaattcagtcat

(19) United States
(12) Patent Application Publication
(10) Pub. No.: US 2024/0271135 A1
(43) Pub. Date: Aug. 15, 2024

(54) COMPOSITION COMPRISING AN RNA THERAPEUTIC TARGETING FAT10 AND USES OF SAME FOR TREATING DISORDERS CHARACTERIZED BY ABNORMAL LIPID ACCUMULATION

(71) Applicant: SHEBA IMPACT LTD., Ramat Gan (IL)

(72) Inventors: Yehuda KAMARI, Ramat Gan (IL); Michal KANDEL-KFIR, Givatayim (IL); Dror HARATS, Ramat-Gan (IL); Dan DOMINISSINI, Kiryat-Ono (IL); Gideon RECHAVI, Tel-Aviv (IL); Aviv SHAISH, Talmei Yechie (IL)

(21) Appl. No.: 18/442,518

(22) Filed: Feb. 15, 2024

Related U.S. Application Data
(63) Continuation of application No. PCT/IL2022/050894, filed on Aug. 17, 2022.
(60) Provisional application No. 63/234,291, filed on Aug. 18, 2021.

(51) Int. Cl. C12N 15/113; A61K 45/06; A61P 3/06

(52) U.S. Cl. CPC C12N 15/113; A61K 45/06; A61P 3/06; C12N 2310/11; C12N 2310/351

(57) ABSTRACT

This disclosure is directed to compositions comprising an RNA therapeutic targeting FAT10 and uses of same.

### CROSS REFERENCE TO RELATED APPLICATIONS

This application is a Bypass Continuation of PCT Patent Application No. PCT/IL2022/050894 having International filing date of Aug. 17, 2022, which claims the benefit of priority of U.S. Provisional Patent Application No. 63/234,291, filed Aug. 18, 2021, the contents of which are all incorporated herein by reference in their entirety.

### REFERENCE TO AN ELECTRONIC SEQUENCE LISTING

The contents of the electronic sequence listing (TSH_013-PCT SEQ. LISTING FINAL.xml; Size: 53,819 bytes; and Date of Creation: Feb. 14, 2024) is herein incorporated by reference in its entirety.

### FIELD OF INVENTION

The present disclosure generally relates to the field of RNA therapeutics, in particular RNA therapeutics that reduce/prevent expression of hepatic FAT10 in a subject in need thereof, in particular subjects suffering from abnormally high amount of lipids in the liver and/or adipose tissue and/or the blood.

### BACKGROUND

Hyperlipidemia is a general term that encompasses diseases and disorders characterized by or associated with elevated levels of lipoproteins in the blood. Hyperlipidemias include hypercholesterolemia, hypertriglyceridemia, combined hyperlipidemia, and elevated lipoprotein a (Lp(a)). Hypercholesterolemia is a particular prevalent form of hyperlipidemia that could be genetic or associated with obesity and type 2 diabetes.

Triglycerides are common types of fats (lipids) that are transported in the blood on lipoproteins and delivered to adipose tissue for storage of energy. They account for about 95 percent of the body's adipose tissue. Abnormally high blood triglyceride levels may be an indication of conditions such as cirrhosis of the liver, underactive thyroid (hypothyroidism), poorly controlled diabetes, or pancreatitis (inflammation of the pancreas). Researchers have identified triglycerides as an independent risk factor for coronary heart disease.

Hypercholesterolemia, with an increase in cholesterol-rich apoB-containing lipoproteins constitutes a major risk for development of atherosclerosis and coronary heart disease (CHD). Clinically, LDL-cholesterol (LDL-C) and Non-HDL-cholesterol (Non-HDL-C) values are the primary targets for cholesterol lowering therapy and are accepted as a valid surrogate therapeutic endpoint in clinical guidelines. Numerous studies have demonstrated that lowering LDL-C or Non-HDL-C levels reduces morbidity and mortality risk from atherosclerotic cardiovascular disease (ASCVD).

Familial hypercholesterolemia (FH) is an inherited disorder of lipid metabolism that predisposes a person to premature onset of cardiovascular disease (CVD). FH can be either an autosomal dominant or an autosomal recessive disease that mostly results from mutations in the low-density lipoprotein receptor (LDLR) but loss of function mutations in apolipoprotein (apo) B or gain of function mutations of PCSK9 can also be manifested with the disease.

Current cholesterol-lowering medications include statins, cholesterol absorption inhibitors, and PCSK9 inhibitors. Statins are a commonly prescribed treatment for cholesterol-lowering. However, despite the availability of such lipid-lowering therapies, many high-risk patients fail to reach their guideline target LDL-C and Non-HDL-C levels.

Accordingly, patients who are not at LDL-C goal, would greatly benefit from alternative cholesterol-lowering therapies, or through use of a combination of therapeutic agents, such as the agents and regimens described herein.

### SUMMARY

The following embodiments and aspects thereof are described and illustrated in conjunction with compositions and methods which are meant to be exemplary and illustrative, not limiting in scope. In various embodiments, one or more of the above-described problems have been reduced or eliminated, while other embodiments are directed to other advantages or improvements.

The present disclosure provides compositions comprising an RNA therapeutic that reduces/prevents expression of FAT10 as well as uses thereof in the treatment of subjects suffering from hypercholesterolemia and obesity-associated co-morbidities.

It was surprisingly found by the inventors of the present invention that FAT10 inhibits age-related hepatic accumulation of triglycerides and cholesterol, suggesting a specific role of FAT10 in promoting lipogenesis in hepatocytes, thus indicating that FAT10 inhibition may be used for treatment of fatty liver.

The herein disclosed composition includes RNA therapeutics specifically directed to inhibit the expression of FAT10. Advantages of RNA therapeutics include: (1) their ability to act on targets that are otherwise “undruggable” for a small molecule or a protein; (2) their rapid and cost-effective development, by comparison to that of small molecules or recombinant proteins; (3) the ability to rapidly alter the sequence of the mRNA construct for personalized treatments or to adapt to an evolving pathogen.

The herein disclosed RNA therapeutics may be conjugated to GalNac molecules, thereby advantageously providing efficient and specific delivery to the liver.

According to some embodiments, there is provided a composition including an RNA therapeutic targeting FAT10 and a suitable carrier.

According to some embodiments, the RNA therapeutic comprises an antisense oligonucleotide (ASO) or an siRNA molecule. According to some embodiments, the RNA therapeutic comprises is an ASO. According to some embodiments, the ASO comprises 18-25 nucleotides having at least 80%, at least 90%, at least 95% or at least 98% sequence complementarity to the FAT10 nucleotide sequence set forth in SEQ ID NO: 2. According to some embodiments, the ASO has at least 90% sequence identity to any of the nucleotide sequence set forth in SEQ ID Nos: 3-5 and SEQ ID Nos: 15-54. Each possibility is a separate embodiment. According to some embodiments, the ASO consists essentially of any of the nucleotide sequence set forth in SEQ ID Nos: 3-5 and SEQ ID Nos: 15-54. Each possibility is a separate embodiment. According to some embodiments, the ASO consists of any of the nucleotide sequence set forth in SEQ ID Nos: 3-5 and SEQ ID Nos: 15-54. Each possibility is a separate embodiment.

According to some embodiments, the RNA therapeutic is conjugated to a GalNac molecule. According to some embodiments, the GalNac molecule is a GalNac trimer. According to some embodiments, the composition is suitable for delivery to the liver.

According to some embodiments, the RNA therapeutic provides liver-specific reduction in FAT-10 levels. According to some embodiments, the FAT-10 levels remain essentially unaltered in non-liver tissue.

According to some embodiments, the composition is for use in the treatment of a disease/disorder associated with an abnormal amount of lipids. According to some embodiments, the lipid associated disease is selected from dyslipidemia, familial hypercholesterolemia, atherosclerotic cardiovascular disease (ASCVD), obesity, type 2 diabetes, hypertension, alcoholic and non-alcoholic fatty liver disease, hepatocellular carcinoma, obesity-associated cancer or any combination thereof. Each possibility is a separate embodiment. According to some embodiments, the lipid associated disease is selected from dyslipidemia, familial hypercholesterolemia, atherosclerotic cardiovascular disease (ASCVD), or any combination thereof. Each possibility is a separate embodiment.

According to some embodiments, there is provided a method for treating, inhibiting preventing and/or ameliorating a disease/disorder associated with an abnormal amount of lipids in a subject, the method comprising administering to the subject an RNA therapeutic targeting a FAT10 RNA molecule of the subject. According to some embodiments, the disease/disorder associated with an abnormal amount of lipids is selected from dyslipidemia, familial hypercholesterolemia, atherosclerotic cardiovascular disease (ASCVD), obesity, type 2 diabetes, hypertension, alcoholic and non-alcoholic fatty liver disease, hepatocellular carcinoma, obesity-associated cancer or any combination thereof. Each possibility is a separate embodiment. According to some embodiments, the lipid associated disease is selected from dyslipidemia, familial hypercholesterolemia, atherosclerotic cardiovascular disease (ASCVD), or any combination thereof. Each possibility is a separate embodiment. According to some embodiments, the hypercholesterolemia is familial hypercholesterolemia.

According to some embodiments, there is provided a method for inhibiting, reducing and/or preventing accumulation of triglycerides and/or cholesterol in a subject in need thereof, the method comprising administering to the subject an RNA therapeutic targeting a FAT10 RNA molecule of the subject

According to some embodiments, the RNA therapeutic comprises an antisense oligonucleotide (ASO) or an siRNA molecule. According to some embodiments, the RNA therapeutic comprises an ASO. According to some embodiments, the ASO comprises 18-25 nucleotides having at least 80%, at least 90%, at least 95% or at least 98% sequence complementarity to the FAT10 nucleotide sequence set forth in SEQ ID NO: 2. According to some embodiments, the ASO has at least 80%, at least 85%, at least 90%, at least 95%, at least 96%, at least 97%, at least 98%, at least 99%, or 100%, sequence identity to any of the nucleotide sequence set forth in SEQ ID Nos: 3-5 and SEQ ID Nos: 15-54. Each possibility is a separate embodiment. According to some embodiments, the ASO consists essentially of any of the nucleotide sequence set forth in SEQ ID Nos: 3-5 and SEQ ID Nos: 15-54. Each possibility is a separate embodiment. According to some embodiments, the ASO consists of any of the nucleotide sequence set forth in SEQ ID Nos: 3-5 and SEQ ID Nos: 15-54. Each possibility is a separate embodiment.

According to some embodiments, the RNA therapeutic is conjugated to a GalNac molecule. According to some embodiments, the GalNac molecule is a GalNac trimer. According to some embodiments, the composition is suitable for delivery to the liver.

According to some embodiments, the RNA therapeutic provides liver-specific reduction in FAT-10 levels. According to some embodiments, the FAT-10 levels remain essentially unaltered in non-liver tissue following administration with the RNA therapeutic.

According to some embodiments, the method further comprises administering to the subject an HMG-COA reductase inhibitor, e.g., statin, a lipid-lowering agent, e.g., ezetimibe, a PCSK9 inhibitor or an ATP citrate-lyase (ACLY) inhibitor.

According to some embodiments, the accumulation of triglycerides and/or cholesterol is hepatic accumulation of triglycerides and/or cholesterol.

According to some embodiments, the familial hypercholesterolemia is heterozygote FH or homozygote FH. Each possibility is a separate embodiment.

According to some embodiments, the disease/disorder is independent of low-density lipoprotein receptor signaling (LDLR independent).

According to some embodiments, there is provided a use of a composition comprising an RNA therapeutic targeting FAT10 for treatment of familial hypercholesterolemia, dyslipidemia, atherosclerotic cardiovascular disease (ASCVD), obesity, type 2 diabetes, hypertension, alcoholic and non-alcoholic fatty liver disease, hepatocellular carcinoma, obesity-associated cancer or any combination thereof.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The invention will now be described in relation to certain examples and embodiments with reference to the following illustrative figures.

FIG. 1A is a histogram chart showing the fold change in FAT10 expression in cells (mouse hepatocyte cell line (FL83B)) treated with 10 ng/ml IFNγ and transfected with various anti-FAT10 ASOs or with saline, as compared to controls cells, which were not treated with IFNγ.

FIG. 1B is a histogram chart showing the fold change in FAT10 expression in cells (mouse hepatocyte cell line (FL83B)) transfected with a subset of the various anti-FAT10 ASOs as compared to control cells treated with saline.

FIG. 1C is a histogram chart showing the fold change in FAT10 expression in cells (mouse hepatocyte cell line (FL83B)) treated with 0.1 ng/ml IFNγ and transfected with a subset of the various anti-FAT10 ASOs or with saline, as compared to controls cells, which were not treated with IFNγ.

FIG. 1D is a histogram chart showing the fold change in FAT10 expression in cells (mouse hepatocyte cell line (FL83B)) transfected with increasing concentrations of a subset of the various anti-FAT10 ASOs or with saline.

FIG. 2A is a histogram chart showing the fold change in FAT10 expression in primary mouse hepatocytes from apoE−/− mice, transfected with increasing concentrations of ASO4 as compared to control primary hepatocytes.

FIG. 2B is a histogram chart showing the fold change in FAT10 expression in primary mouse hepatocytes from apoE−/− mice, transfected with increasing concentrations of ASO6 as compared to control primary hepatocytes.

FIG. 2C is a histogram chart showing the fold change in FAT10 expression in primary mouse hepatocytes from apoE−/− mice, transfected with increasing concentrations of ASO11 as compared to control primary hepatocytes.

FIG. 3A shows luciferase intensity obtained for three human FAT10-ASOs using a human FAT10 luciferase reporter-based platform (psi-check).

FIG. 3B is a histogram chart showing the fold change in FAT10 expression in human hepatocyte cell line (HEPG2), transfected with increasing concentrations of FAT10 ASOs and treated with IFNγ+TNFα, as compared to control cells.

FIG. 3C is a western blot of human FAT10 protein in human hepatocyte cell line (HEPG2), transfected with three human FAT10 ASOs and treated with of IFNγ+TNFα, as compared to control cells.

FIG. 4A is a histogram chart showing the fold change in FAT10 expression in FL83B mouse hepatocyte cell line (WT) compared to FL83B cells with knockdown of FAT10 (ShFAT10) either not treated (NT) or treated with TNFα.

FIG. 4B shows the expression level of SREBP2 as well as its target genes in FL83B mouse hepatocyte cell line (white column) and FL83B cells with knockdown of FAT10 (black columns) grown with either normal growth fetal calf serum (FCS) versus lipoprotein-deficient serum plus Compactin and Mevalonate (LPDS+CM).

FIG. 4C shows the expression level of SREBP1c as well as its target genes in FL83B mouse hepatocyte cell line (white column) and FL83B cells with knockdown of FAT10 (black columns) grown with either normal growth fetal calf serum (FCS) versus lipoprotein-deficient serum plus Compactin and Mevalonate (LPDS+CM).

FIG. 5A is a histogram chart showing the fold change in FAT10 expression in human hepatocyte cells line (HEPG2) (white bars) compared to HEPG2 following CRISPR mediated FAT10 KD (black bars) and grown with either normal growth fetal calf serum (FCS) versus lipoprotein-deficient serum plus Compactin and Mevalonate (LPDS+CM).

FIG. 5B shows the expression level of PCSK9 in human hepatocyte cells line (HEPG2) (white bars) compared to HEPG2 following CRISPR mediated FAT10 KD (black bars) and grown with either normal growth fetal calf serum (FCS) versus lipoprotein-deficient serum plus Compactin and Mevalonate (LPDS+CM).

FIG. 5C shows the expression level of HMGCR in human hepatocyte cells line (HEPG2) (white bars) compared to HEPG2 following CRISPR mediated FAT10 KD (black bars) and grown with either normal growth fetal calf serum (FCS) versus lipoprotein-deficient serum plus Compactin and Mevalonate (LPDS+CM).

FIG. 5D shows the expression level of LDLR in human hepatocyte cells line (HEPG2) (white bars) compared to HEPG2 following CRISPR mediated FAT10 KD (black bars) and grown with either normal growth fetal calf serum (FCS) versus lipoprotein-deficient serum plus Compactin and Mevalonate (LPDS+CM).

FIG. 6A is a histogram chart showing the fold change in FAT10 expression in liver of apoE−/− mice undergoing three weekly s.c. injections with a FAT10 ASO (ASO4 and ASO11) or with saline as control.

FIG. 6B shows a graph of body weight in apoE−/− mice injected with a FAT10 ASO (ASO4 and ASO11) or with saline as control.

FIG. 6C is a histogram chart of a SGOT liver function test in apoE−/− mice injected with a FAT10 ASO (ASO4 and ASO11) or with saline as control.

FIG. 6D is a histogram chart of a SGPT liver function test in apoE−/− mice injected with a FAT10 ASO (ASO4 and ASO11) or with saline as control.

FIG. 6E is a histogram chart of epididymal white adipose tissue weight (eWAT (gr)) of apoE−/− mice injected with a FAT10 ASO (ASO4 and ASO11) or with saline as control.

FIG. 6F is a histogram chart of total plasma cholesterol (mg/dl) in apoE−/− mice injected with a FAT10 ASO (ASO4 and ASO11) or with saline as control.

FIG. 6G is a histogram chart of LDLR mRNA expression levels in apoE−/− mice injected with a FAT10 ASO (ASO4 and ASO11) or with saline as control.

FIG. 6H is a histogram chart of PCSK9 mRNA expression levels in apoE−/− mice injected with a FAT10 ASO (ASO4 and ASO11) or with saline as control.

FIG. 6I is a histogram chart of HMGCR mRNA expression levels in apoE−/− mice injected with a FAT10 ASO (ASO4 and ASO11) or with saline as control.

FIG. 6J is a histogram chart of PCSK9 protein plasma levels in apoE−/− mice injected with a FAT10 ASO (ASO4 and ASO11) or with saline as control.

FIG. 7A: is a histogram chart of total lipids weight per g liver tissue in young (4 months) and old (18 months) non-fasting female WT and FAT10−/− mice fed regular chow diet.

FIG. 7B is a histogram chart of thin layer chromatography (TLC) analysis of lipids extracted from livers of young (4 months) non-fasting female WT and FAT10−/− mice fed regular chow diet. CE: cholesterol ester; TG: triacylglyceride; FFA: free fatty acid, DG: diacylglyceride; Ch: free cholesterol; PL: phospholipid.

FIG. 7C is a histogram chart of thin layer chromatography (TLC) analysis of lipids extracted from livers of old (18 months) non-fasting female WT and FAT10−/− mice fed regular chow diet. CE: cholesterol ester; TG: triacylglyceride; FFA: free fatty acid, DG: diacylglyceride; Ch: free cholesterol; PL: phospholipid.

FIG. 8A is a histogram chart of % liver to body weight of WT and FAT10−/− mice fed regular chow or fructose diet.

FIG. 8B is a histogram chart of total liver lipids of WT and FAT10−/− mice fed regular chow or fructose diet.

FIG. 8C is a histogram chart of liver TG accumulation in WT and FAT10−/− mice fed regular chow or fructose diet.

FIG. 8D is a histogram chart of SREBP1c and its target genes FASN, SCD1, ELOVL6, ACCa, and ChREBP in the liver of WT mice fed regular chow or fructose diet.

FIG. 8E is a histogram chart of SREBP1c and its target genes FASN, SCD1, ELOVL6, ACCa, and ChREBP in the liver of FAT10−/− mice fed regular chow or fructose diet.

FIG. 9A is a histogram chart of total body weight in WT and FAT10−/− mice fed with regular chow or high fat diet (HFD).

FIG. 9B is a histogram chart of total body fat in WT and FAT10−/− mice fed with regular chow or high fat diet (HFD).

FIG. 9C is a histogram chart of lean body weight in WT and FAT10−/− mice fed with regular chow or high fat diet (HFD).

FIG. 9D is a histogram chart of plasma cholesterol levels in WT and FAT10−/− mice fed with regular chow or high fat diet (HFD).

FIG. 9E is a histogram chart of plasma apoB levels in WT and FAT10−/− mice fed with regular chow or high fat diet (HFD).

FIG. 9F is a histogram chart of plasma PCSK9 levels in WT and FAT10−/− mice fed with regular chow or high fat diet (HFD).

FIG. 9G is a histogram chart of SREBP2 and SQS mRNA expression levels in WT and FAT10−/− mice fed with regular chow or high fat diet (HFD).

FIG. 9H is a histogram chart of PCSK9 and Insig1 mRNA expression levels in WT and FAT10−/− mice fed with regular chow or high fat diet (HFD).

FIG. 9I is a histogram chart of SREBP1c, ACC and SCD1 mRNA expression levels in WT and FAT10−/− mice fed with chow or high fat diet (HFD).

FIG. 9J is a histogram chart of PNPLA3 mRNA expression levels in WT and FAT10−/− mice fed with regular chow or high fat diet (HFD).

FIG. 10A is a histogram chart of SREBP2 and its target genes PCSK9, HMGCS1, INSIG1, ACAT2, SQS, LDLR, ACLY, and HMGCR mRNA expression levels in apoE−/− and apoE−/− FAT10−/− double knockout (DKO) mice.

FIG. 10B is a histogram chart of total plasma cholesterol levels in WT, FAT10−/−, apoE−/− and apoE−/− FAT10−/− (DKO) mice.

FIG. 10C is a line chart of plasma lipoprotein cholesterol on VLDL, IDL/LDL, and HDL FPLC fractions in apoE−/− and apoE−/− FAT10−/− (DKO) mice.

FIG. 10D is a histogram chart of plasma apoB levels in apoE−/− and apoE−/− FAT10−/− (DKO) mice.

FIG. 10E is a histogram chart of plasma PCSK9 levels in apoE−/− and apoE−/− FAT10−/− (DKO) mice.

FIG. 10F is a dot plot representation of aortic sinus atherosclerotic plaque area in apoE−/− and apoE−/− FAT10−/− (DKO) mice.

FIG. 10G is a representative image of aortic sinus atherosclerotic plaque area in apoE−/− and apoE−/− FAT10−/− (DKO) mice.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical object of the article. By way of example, “an element” means one element or more than one element.

The term “about” when referring to a measurable value such as an amount, a temporal duration, and the like, is meant to encompass variations of +20% or in some instances+10%, or in some instances+5%, or in some instances+1%, or in some instances+0.1% from the specified value, as such variations are appropriate to perform the disclosed methods.

The terms “subject”, “patient” or “individual” generally refer to a human, although the methods of the invention are not necessarily limited to humans and should be useful in other mammals.

As used herein, a “nucleotide” comprises a nitrogenous base, a sugar molecule, and a phosphate group. A nucleic acid may include naturally occurring nucleosides (e.g., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, locked nucleic acids, arabinose, and hexose). According to some embodiments, the sugar and/or phosphate groups may be modified to include a peptide bond, so as to obtain a Peptide Nucleotide Acid (PNA).

As used herein, the term “RNA” or “RNA molecule” or “ribonucleic acid molecule” refers to a polymer of ribonucleotides. The term “DNA” or “DNA molecule” or deoxyribonucleic acid molecule” refers to a polymer of deoxyribonucleotides. DNA and RNA can be synthesized naturally (e.g., by DNA replication or transcription of DNA or RNA, respectively). DNA and RNA can also be chemically synthesized. RNA can be post-transcriptionally modified. The terms “target mRNA” and “target transcript” are synonymous as used herein.

As used herein, the term “RNA therapeutic” refers to RNA drugs that are based on one of two main approaches: (1) antisense RNA (RNAi), where short oligonucleotides recognize and hybridize to complementary sequences in endogenous RNA transcripts and alter their processing; and (2) message RNA (mRNA), where mRNAs encoding certain peptides or proteins elicit their transient expression in the cytoplasm. Non-limiting examples of RNA therapeutics include antisense oligonucleotides (ASO), aptamers, small interfering RNAs, microRNAs, and messenger RNA.

As used herein, the term “Antisense Oligonucleotide” and “ASO” refer to short single-stranded DNA, phosphorothioate DNA, RNA analogs, conformationally restricted nucleosides (locked nucleic acids, LNA), or morpholino phosphorodiamidate oligonucleotides complementary to a certain region of RNA that they are meant to target. The modifications in backbone, and sugar molecules give antisense oligos more affinity and stability. There are two classes of ASOs: RNase H-dependent ASO and RNase H-independent (steric block) ASO. Steric block ASOs physically inhibit or prevent translation or splicing, and can be engineered to either prevent polyadenylation, inhibit or enhance translation, or alter splicing. The RNase H-dependent ASO is more commonly used and is dependent on the endogenous RNase H enzyme that hydrolyzes the RNA strand of an RNA/DNA duplex. The RNase H-dependent ASOs are generally more efficient in knockdown of gene expression than RNase H-independent ASOs.

As used herein, the term “RNA interference” (“RNAi”) refers to selective intracellular degradation of RNA (also referred to as gene silencing). As used herein a RNAi molecule may collectively refer to small interfering RNAs and short hairpin RNA.

As used herein, the term “small interfering RNA” (“siRNA”), also referred to in the art as “short interfering RNAs,” refers to an RNA (or RNA analog) comprising between about 10-60 or 15-25 nucleotides (or nucleotide analogs) that is capable of directing or mediating RNA interference. Generally, as used herein the term “siRNA” refers to double stranded siRNA (as compared to single stranded or antisense RNA). In certain embodiments, the 3′ end of the RNAi molecules may include additional nucleotides that create an overhang, such as “TT”. Small interfering RNAs (siRNAs) are small non-coding RNA duplexes that originate from precursor siRNAs. The latter are either transcribed or artificially introduced and range from 30 bp to more than 100 bp. The precursor siRNA duplex is processed by the endogenous Dicer enzyme into 20-30 bp long siRNA with two base overhangs in the 3′ region, which interacts with the endogenous RNA-induced silencing complex (RISC) to elicit RNA interference (RNAi). The endonuclease argonaute 2 (AGO2) component of the RISC cleaves the sense strand, leaving intact the antisense strand, which guides the active RISC to its target mRNA. Then AGO2 cleaves the phosphodiester backbone of the target mRNA. The antisense strand is usually fully complementary to the coding region of the target mRNA, therefore siRNA knocks down one specific target gene

As used herein, the term “short hairpin RNA” (“shRNA”) refers to an siRNA (or siRNA analog) precursor that is folded into a hairpin structure and contains a single stranded portion of at least one nucleotide (a “loop”), e.g., an RNA molecule that contains at least two complementary portions hybridized or capable of hybridizing to form a double-stranded (duplex) structure sufficiently long to mediate RNAi (as described for siRNA duplexes), and at least one single-stranded portion, typically between approximately 1 and 10 nucleotides in length that forms a loop connecting the regions of the shRNA that form the duplex portion. The duplex portion may, but typically does not, contain one or more mismatches and/or one or more bulges consisting of one or more unpaired nucleotides in either or both strands. Without wishing to be bound by theory, shRNAs are thought to be processed into siRNAs by the conserved cellular RNAi machinery. shRNAs are capable of inhibiting expression of a target transcript that is complementary to a portion of the shRNA (referred to as the antisense or guide strand of the shRNA). In general, the features of the duplex formed between the guide strand of the shRNA and a target transcript are similar to those of the duplex formed between the guide strand of an siRNA and a target transcript. In certain embodiments of the invention the 5′ end of an shRNA has a phosphate group while in other embodiments it does not. In certain embodiments of the invention the 3′ end of an shRNA has a hydroxyl group.

As used herein, the term “microRNAs” and miRNAs refer to small non-coding RNA molecules that regulate the expression of multiple mRNAs by blocking translation or promoting degradation of the target mRNAs. This class of non-coding RNAs are transcribed from genomic DNA as primary miRNAs (pri-miRNAs). The latter adopt a loop structure with interspersed mismatches and are cleaved by Drosha to a 70-100 bp precursor miRNAs (pre-miRNAs), before leaving the nucleus. Exportin 5 transports the pre-miRNAs to the cytoplasm, where Dicer processes them into 18-25 bp RNA duplexes with two base overhangs in the 3′ region. These structures are now referred to as miRNAs. The miRNA is then loaded into the RISC to form a miRISC complex. The miRNA duplex unwinds to release the sense strand. The antisense strand guides the miRISC. Hybridization usually occurs at 2-7 bases of the 5′ end of miRNA and the 3′ UTR of the target mRNA. The target mRNA is inhibited via translational repression, degradation or cleavage. The miRNA-based therapeutics could be categorized into two types: miRNAs mimics and miRNAs inhibitors. The former are double-stranded RNA molecules that mimic miRNAs, while the latter are single-stranded RNA oligos designed to interfere with miRNAs.

As used herein, the term “RNAi-inducing vector” includes a vector whose presence within a cell results in transcription of one or more RNAs that self-hybridize or hybridize to each other to form an RNAi molecule. In various embodiments of the invention this term encompasses plasmids, e.g., DNA vectors (whose sequence may comprise sequence elements derived from a virus), or viruses, (other than naturally occurring viruses or plasmids that have not been modified by the hand of man), whose presence within a cell results in production of one or more RNAs that self-hybridize or hybridize to each other to form an RNAi molecule. In general, the vector comprises a nucleic acid operably linked to expression signal(s) so that one or more RNA molecules that hybridize or self-hybridize to form an RNAi molecule is transcribed when the vector is present within a cell. Use of the term “induce” is not intended to indicate that the RNAi agent necessarily activates or upregulates RNAi in general but simply indicates that presence of the vector within a cell results in production of an RNAi agent within the cell, leading to an RNAi-mediated reduction in expression of an RNA to which the agent is targeted.

An RNAi-inducing entity is considered to be targeted to a target transcript for the purposes described herein if (1) the agent comprises a strand that is substantially complementary to the target transcript over 15-29 nucleotides, e.g., 15, more preferably at least about 17, yet more preferably at least about 18 or 19 to about 21-23 or 24-29 nucleotides. For example, in various embodiments of the invention the agent comprises a strand that has at least about 70%, preferably at least about 80%, 84%, 89%, 90%, 91%, 92%, 93%, 94%, 95%, 96%, 97%, 98%, 99%, or 100% precise sequence complementarity with the target transcript over a window of evaluation between 15-29 nucleotides in length, e.g., over a window of evaluation of at least 15, more preferably at least about 17, yet more preferably at least about 18 or 19 to about 21-23 or 24-29 nucleotides in length; or (2) one strand of the RNAi agent hybridizes to the target transcript under stringent conditions for hybridization of small (<50 nucleotide) RNA molecules in vitro and/or under conditions typically found within the cytoplasm or nucleus of mammalian cells.

According to some embodiments, the RNA therapeutic may be stabilized. As used herein a “stabilized RNA therapeutic” may refer to RNA molecules that can contain stabilizing elements, including, but not limited to a 5′-cap structure or a 3′-poly(A) tail.

The 5′-capping of polynucleotides may be completed concomitantly during the in vitro-transcription reaction e.g. using the following chemical RNA cap analogs to generate the 5′-guanosine cap structure according to manufacturer protocols: 3′-O-Me-m7G(5′)ppp(5′) G [the ARCA cap]; G(5′)ppp(5′)A; G(5′)ppp(5′)G; m7G(5′)ppp(5′)A; m7G(5′)ppp(5′)G (New England BioLabs, Ipswich, Mass.). 5′-capping of modified RNA may be completed post-transcriptionally using a Vaccinia Virus Capping Enzyme to generate the “Cap 0” structure: m7G(5′)ppp(5′)G (New England BioLabs, Ipswich, Mass.). Cap 1 structure may be generated using both Vaccinia Virus Capping Enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase. Each possibility is a separate embodiment. According to some embodiments, the capping comprises a 7-methylguanosine cap (m7G) or a m7G-analog.

According to some embodiments, the RNA therapeutic, e.g., the ASO may be conjugated with N-acetylgalactosamine (GalNAc), preferably a GalNac trimer, for delivery into the liver. Tris-GalNAc binds to the Asialoglycoprotein receptor that is predominantly expressed on liver hepatocytes.

According to some embodiments, the RNA therapeutic may be encapsulated in lipid nanoparticles (LNPs). The LNP-encapsulated siRNAs are approximately 100 nm in diameter and have neutral surface charge, which allows effective delivery to the liver parenchyma via the sinusoidal fenestrae. Upon internalization and transfer into late endosomal compartments, the change in endosomal pH causes the cationic lipids in the LNP to undergo phase transition, forming an inverted hexagonal phase, a nonbilayer lipid structure that induces membrane permeability and LNP disintegration. According to some embodiments, the RNA therapeutic may be encapsulated in vitamin A-modified nanoparticles.

According to some embodiments, the RNA therapeutic may be cholesterol conjugated. According to some embodiments, the RNA therapeutic may be tocopherol-conjugated.

According to some embodiments, the RNA therapeutic may be modified to enhance stability, reduce degradation and/or promote tissue specific delivery. The modifications can be made to the backbone, sugar, or nucleobases of RNA therapeutic.

Non-limiting examples of modifications that may be applied to enhance nuclease stability include: modifications at the 2′ position of the furanose ring in natural nucleic acids such as 2′-O-methyl (OMe), 2′-fluoro (F), and 2′-O-methoxyethyl (MOE) RNA. Conformational restriction of the furanose ring into the C3′-endo sugar pucker generating locked nucleic acid (LNA) and constrained ethyl (cEt), phosphorodiamidate morpholinos (PMOs).

According to some embodiments, the RNA therapeutic may include a phosphorothioate (PS) backbone modification in which the nonbridging oxygen atoms of the natural phosphodiester linkage is replaced with a sulfur atom. Without being bound by any theory, the PS-modified RNA therapeutic may bind to plasma proteins such as albumin, thereby facilitating its distribution to tissues peripheral from the site of injection including the liver.

Unless otherwise stated, nucleotide sequences in the text of this specification are given, when read from left to right, in the 5′ to 3′ direction. The nomenclature of nucleotides and amino acid symbols used herein is that required by Title 37 of the United States Code of Federal Regulations § 1.822 and set forth in the tables in WIPO Standard ST.26 (2022), Annex 1, Tables 1 and 3.

The terms “upstream” and “downstream”, as used herein refers to a relative position in a nucleotide sequence, such as, for example, a DNA sequence or an RNA sequence. As well known, a nucleotide sequence has a 5′ end and a 3′ end, so called for the carbons on the sugar (deoxyribose or ribose) ring of the nucleotide backbone. Hence, relative to the position on the nucleotide sequence, the term downstream relates to the region towards the 3′ end of the sequence. The term upstream relates to the region towards the 5′ end of the strand.

As used herein, the term “homolog” may refer to a polynucleotide having substantially from about 70% to about 99% sequence identity, or more preferably from about 80% to about 99% sequence identity, or most preferable from about 90% to about 99% sequence identity, to about 99% sequence identity, to the referent nucleotide sequences of a referent polynucleotide molecule. Each possibility is a separate embodiment.

As used herein, the term “sequence identity”, “sequence similarity” or “homology” is used to describe sequence relationships between two or more nucleotide sequences. The percentage of “sequence identity” between two sequences is determined by comparing two optimally aligned sequences. A sequence that is identical at every position in comparison to a reference sequence is said to be identical to the reference sequence and vice-versa. A first nucleotide sequence when observed in the 5′ to 3′ direction is said to be a “complement” of, or complementary to, a second or reference nucleotide sequence observed in the 3′ to 5′ direction if the first nucleotide sequence exhibits complete complementarity with the second or reference sequence. As used herein, nucleic acid sequence molecules are said to exhibit “complete complementarity” when every nucleotide of one of the sequences read 5′ to 3′ is complementary to every nucleotide of the other sequence when read 3′ to 5′. A nucleotide sequence that is complementary to a reference nucleotide sequence will exhibit a sequence identical to the reverse complement sequence of the reference nucleotide sequence. These terms and descriptions are well defined in the art and are easily understood by those of ordinary skill in the art. According to some embodiments, the term sequence homology refers to 70%, 80%, 85%, 90% or 95% identity. Each possibility is a separate embodiment.

As referred to herein, the term “complementarity” is directed to base pairing between strands of nucleic acids. As known in the art, each strand of a nucleic acid may be complementary to another strand in that the base pairs between the strands are non-covalently connected via two or three hydrogen bonds. Two nucleotides on opposite complementary nucleic acid strands that are connected by hydrogen bonds are called a base pair. According to the Watson-Crick DNA base pairing, adenine (A) forms a base pair with thymine (T) and guanine (G) with cytosine (C). In RNA, thymine is replaced by uracil (U). The degree of complementarity between two strands of nucleic acid may vary, according to the number (or percentage) of nucleotides that form base pairs between the strands. For example, “100% complementarity” indicates that all the nucleotides in each strand form base pairs with the complement strand. For example, “95% complementarity” indicates that 95% of the nucleotides in each strand from base pair with the complement strand. The term sufficient complementarity may include any percentage of complementarity from about 30% to about 100%.

As used herein, the term “consist essentially”, refers to the sequences of the ASO nucleotide as they are set forth in any one of the SEQ ID NO, and means to exclude additional, unrecited elements, therefore limiting the scope of the nucleic acid residues of the ASO of the invention only to those described in WIPO Standard ST.26 (2022), Annex 1, table 1, excluding, for example, nucleotide analogs and modified nucleotides,

As used herein, the term “administration” to a subject can be carried out using known procedures, at dosages and for periods of time effective to provide the desired effect. An effective amount of the therapeutic compound necessary to achieve a therapeutic effect may vary according to factors such as the age, sex, and weight of the subject, and the ability of the therapeutic compound to treat the foreign agents in the subject. Dosage regimens can be adjusted to provide the optimum therapeutic response.

Administration as used herein encompass both one subject providing administering the herein disclosed RNAi molecules or compositions comprising same to another subject as well as self-administration.

“Administering” includes routes of administration which allow the compositions of the invention to perform their intended function. A variety of routes of administration are possible including, but not necessarily limited to parenteral (e.g., intravenous, intraarterial, intramuscular, subcutaneous injection), oral (e.g., dietary), inhalation (e.g., aerosol to lung), topical, nasal, rectal, or via slow releasing microcarriers depending on the disease or condition to be treated. Inhalation and nasal and/or buccal spraying are preferred modes of administration. Formulation of the compound to be administered will vary according to the route of administration selected (e.g., solution, emulsion, gels, aerosols, capsule). An appropriate composition comprising the compound to be administered can be prepared in a physiologically acceptable vehicle or carrier and optional adjuvants and preservatives. For solutions or emulsions, suitable carriers include, for example, aqueous or alcoholic/aqueous solutions, emulsions or suspensions, including saline and buffered media, sterile water, creams, ointments, lotions, oils, pastes and solid carriers.

According to some embodiments, the term “carrier” may refer to the part of the composition enabling its delivery. According to some embodiments, the carrier may be water or saline. According to some embodiments, the carrier may be an oil. According to some embodiments, the carrier may be a surfactant. As used herein, the term, a “pharmaceutically acceptable carrier” includes any and all solvents, dispersion media, coatings, antibacterial and antifungal agents, isotonic and absorption delaying agents, and the like which are compatible with the activity of the compound and are physiologically acceptable to the subject. An example of a pharmaceutically acceptable carrier is buffered normal saline (0.15M NaCl). The use of such media and agents for pharmaceutically active substances is well known in the art. Except insofar as any conventional media or agent is incompatible with the therapeutic compound, use thereof in the compositions suitable for pharmaceutical administration is contemplated. Supplementary active compounds can also be incorporated into the compositions.

According to some embodiments, the carrier may be a nanoparticle. As used herein, the term “nanoparticle” refers to small particles, typically in the range of between about 1 to 1 about 00 nanometers in size.

According to some embodiments, the nanoparticle may be a lipid-based nanoparticle. According to some embodiments, the lipid-based nanoparticle may be a Liposome. Liposomes, as used herein are spherical vesicles having at least one phospholipid bilayer enclosing an aqueous core.

Liposomes have an inherent advantage in that they mimic cell membrane composition and can encapsulate RNAs when combined with cationic lipids. Positively charged lipids can electrostatically interact with negatively charged RNAs to form complexes of RNA and liposomes. In this way, RNA is encapsulated within liposomes. Cationic lipids such as, DOTMA (1,2-di-O-octadecenyl-3-trimethylammonium-propane) and DOTAP (1,2-dioleoyl-3-trimethylammonium-propane) readily form complexes with negatively charged RNA. The use of cholesterol modified lipid makes the resulting complex more stable and improves transfection.

According to some embodiments, the nanoparticle may be a polymer-based nanoparticle. Polymer nanomaterials normally refer to synthetic compounds made of a handful of base units that come together to form complex structures. These materials usually include synthetic polymers such as PLGA [ploy(lactic-co-glycolic acid)], PLA (polylactic acid), chitosan, gelatin, polycaprolactone, and poly-alkyl-cyanoacrylates. These materials have the virtue of a long shelf life; the ability to encapsulate hydrophilic and hydrophobic compounds and proteins; and the capability for tuned delivery of therapeutic compounds. Polymers can be synthesized to create injectable nanoparticles that can be delivered as intravenous injections or administered as intramuscular, subdermal or intraperitoneal drug depots that degrade over a period of months or weeks.

According to some embodiments, the RNA therapeutic may be administered as naked RNA.

According to some embodiments, the composition may include one or more additional ingredients. “Additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Each possibility is a separate embodiment.

FAT10, also referred to as ubiquitin D, UBD and GABBR1-3 refers to a ubiquitin-like modifier that directly targets proteins for proteasomal degradation or interacts with proteins to modify their activity and/or their subcellular localization. It is encoded in the major histocompatibility complex and is synergistically inducible by tumor necrosis factor alpha and gamma interferon. It is composed of two ubiquitin-like domains and possesses a free C-terminal diglycine motif that is required for the formation of FAT10 conjugates.

The amino acid sequence of human FAT10 is set forth in SEQ ID NO: 1
recited below:
MAPNASCLCVHVRSEEWDLMTFDANPYDSVKKIKEHVRSKTKVPVQDQVLLLGSKILKPRRS
LSSYGIDKEKTIHLTLKVVKPSDEELPLELVESGDEAKRHLLQVRRSSSVAQVKAMIETKTG
IIPETQIVTCNGKRLEDGKMMADYGIRKGNLLFLACYCIGG
The nucleotide sequence of human FAT10 is set forth in SEQ ID NO: 2
recited below:
gtctctggtttctggccccttgtctgcagagatggctcccaatgcttcctgcctctgtgtgc
atgtccgttccgaggaatgggatttaatgacctttgatgccaacccatatgacagcgtgaaa
aaaatcaaagaacatgtccggtctaagaccaaggttcctgtgcaggaccaggttcttttgct
gggctccaagatcttaaagccacggagaagcctctcatcttatggcattgacaaagagaaga
ccatccaccttaccctgaaagtggtgaagcccagtgatgaggagctgcccttgtttcttgtg
gagtcaggtgatgaggcaaagaggcacctcctccaggtgcgaaggtccagctcagtggcaca
agtgaaagcaatgatcgagactaagacgggtataatccctgagacccagattgtgacttgca
atggaaagagactggaagatgggaagatgatggcagattacggcatcagaaagggcaactta
ctcttcctggcatgttattgtattggagggtgaccaccctgggcatggggtgttggcagggg
tcaaaaagcttatttcttttaatctcttactcaacgaacacatcttctgatgatttcccaaa
attaatgagaatgagatgagtagagtaagatttgggtgggatgggtaggatgaagtatattg
cccaactctatgtttctttgattctaacacaattaattaagtgacatgatttttactaatgt
attactgagactagtaaataaatttttaaggcaaaatagagcattcaaagccagcttggaat
ttaattctgtcttgataccttgttatttatgcaaaaactcctatctcctttcctttatgaca
agagagtaagttttaggttgggatcc

According to some embodiments, the RNA therapeutics comprises 18-25 ribonucleotides with at least 80%, at least 85% at least 90%, at least 92%, at least 95% at least 98% or 100% sequence complementarity to a string of consecutive ribonucleotides of FAT10, as set forth in SEQ ID NO: 2. Each possibility is a separate embodiment.

According to some embodiments, the RNA therapeutics comprises an ASO having the sequence set forth in SEQ ID NO: 3 (TGGTCCTGCACAGGAACCTT).

According to some embodiments, the RNA therapeutics comprises an ASO having the sequence set forth in SEQ ID NO: 4 (AGGCTTCTCCGTGGCTTTAA).

According to some embodiments, the RNA therapeutics comprises an ASO having the sequence set forth in SEQ ID NO: 5 (GGTGCCTCTTTGCCTCATCA).

According to some embodiments, the RNA therapeutics comprises an ASO having the sequence set forth in any one of SEQ ID NOs: 15-54. Each possibility is a separate embodiment.

According to some embodiments, the composition is suitable for the treatment of dyslipidemia. As used herein, the term “dyslipidemia” refers to disorders characterized by an abnormal amount of lipids (e.g., triglycerides, cholesterol and/or fat phospholipids) in the blood of the subject suffering therefrom. Non-limiting examples of dyslipidemia include Hypercholesterolemia (cholesterol), Hypertriglyceridemia (glycerides), Hyperlipoproteinemia (lipoproteins, usually LDL but also VLDL and IDL), combined hyperlipidemia (both LDL and triglycerides) and Honemia: chylomicrons. Each possibility is a separate embodiment.

According to some embodiments, the composition is suitable for the treatment of Hypercholesterolemia. As used herein, the term “Hypercholesterolemia” also called high cholesterol refers to presence of high levels of cholesterol in the blood.

According to some embodiments, the composition is suitable for the treatment of aging-related accumulation of lipids including cholesterol ester and triacylglyceride. Each possibility is a separate embodiment

According to some embodiments, the composition is suitable for attenuating liver weight gain, increase in total liver lipids, and triacylglyceride accumulation, associated with fatty liver. Each possibility is a separate embodiment.

According to some embodiments, the composition is suitable for attenuating induction of SREBP1c expression and expression of its target genes including FASN, SCD1, ELOVL6, and ACCa, associated with fatty liver. Each possibility is a separate embodiment.

According to some embodiments, the composition is suitable for preventing hepatocytes from accumulating fatty acids in the liver.

According to some embodiments, the composition is suitable for treating fatty liver.

According to some embodiments, the composition is suitable for attenuating body weight gain, and increase in total body fat associated with obesity. Each possibility is a separate embodiment

According to some embodiments, the composition is suitable for attenuating increase in plasma levels of cholesterol, apoB, and PCSK9, associated with obesity. Each possibility is a separate embodiment

According to some embodiments, the composition is suitable for attenuating hepatic activation of SREBP2, SREBP1c, and their target genes including SQS, PCSK9, INSIG1, ACC, SCD1, and PNPLA3 in vivo, associated with obesity. Each possibility is a separate embodiment.

According to some embodiments, the composition is suitable for treating obesity.

According to some embodiments, the composition is suitable for attenuating hepatic expression of SREBP2 and activation of its target genes including PCSK9, HMGCS1, INSIG1, ACAT2, SQS, LDLR, ACLY, and HMGCR, associated with hypercholesterolemia and/or atherosclerosis. Each possibility is a separate embodiment.

According to some embodiments, the composition is suitable for reducing levels of total plasma cholesterol and of cholesterol carried by apoB-containing lipoproteins VLDL, IDL and LDL, but not HDL, associated with hypercholesterolemia and/or atherosclerosis. Each possibility is a separate embodiment.

According to some embodiments, the composition is suitable for attenuating the expression of plasma apoB levels and plasma PCSK9 levels associated with hypercholesterolemia and/or atherosclerosis. Each possibility is a separate embodiment.

According to some embodiments, the composition is suitable for attenuating aortic sinus atherosclerotic plaque area associated with hypercholesterolemia and/or atherosclerosis.

According to some embodiments, the composition is suitable for treating hypercholesterolemia.

According to some embodiments, the composition is suitable for treating atherosclerosis.

According to some embodiments, the composition is suitable for reducing the levels of plasma cholesterol and of cholesterol carried by apoB-containing lipoproteins VLDL, IDL and LDL. Each possibility is a separate embodiment.

According to some embodiments, the composition is suitable for lowering plasma cholesterol levels and could be utilized for treating patients with heterozygote and homozygote FH. Each possibility is a separate embodiment.

According to some embodiments, reducing the levels of plasma cholesterol or of cholesterol carried by apoB-containing lipoproteins VLDL, IDL and LDL is independent of LDL receptor (LDLR) clearance into the liver. Each possibility is a separate embodiment

The terms “Familial hypercholesterolemia” and “FH” refer to a genetic disorder characterized by high cholesterol levels, specifically very high levels of low-density lipoprotein (LDL, “bad cholesterol”), in the blood and early cardiovascular disease. The most common mutations diminish the number of functional LDL receptors in the liver. Since the underlying body biochemistry is slightly different in individuals with FH, their high cholesterol levels are less responsive to the kinds of cholesterol control methods such as dietary modification and statin tablets. About 1 in 100 to 200 people have mutations in the LDLR gene that encodes the LDL receptor protein, which normally removes LDL from the circulation, or apolipoprotein B (ApoB), which is the part of LDL that binds with the receptor. People who have one abnormal copy (are heterozygous) of the LDLR gene may develop cardiovascular disease prematurely at the age of 30 to 40. Having two abnormal copies (being homozygous) may cause severe cardiovascular disease in childhood. Heterozygous FH is a common genetic disorder, inherited in an autosomal dominant pattern, occurring in 1:250 people in most countries; homozygous FH is much rarer, occurring in about 1 in 1,000,000 people. As used herein, according to some embodiments, the familial hypercholesterolemia is heterozygote FH; according to some embodiments, the familial hypercholesterolemia is homozygote FH.

The terms “Atherosclerotic cardiovascular disease” and “ASCVD” refers to a disease caused by plaque buildup in arterial walls and refers to conditions that include: Coronary Heart Disease (CHD), such as myocardial infarction, angina, and coronary artery stenosis, Carotid artery stenosis causing ischemic cerebrovascular accident (CVA) and peripheral arterial disease (PAD) causing intermittent claudication and leg amputations. Atherosclerosis in which the wall of the artery develops abnormalities, called lesions. A major risk factor for ASCVD is abnormally elevated blood cholesterol levels,

The term “obesity” refers to a medical condition in which excess body fat has accumulated to an extent that it may have a negative effect on health. People are generally considered obese when their body mass index (BMI), a measurement obtained by dividing a person's weight by the square of the person's height is above 30 kg/m2.

The terms “Type 2 diabetes”, “T2D” and adult-onset diabetes, refers to a form of diabetes that is characterized by high blood sugar, insulin resistance, and relative lack of insulin which is acquired primarily as a result of lifestyle and genetic predispositions.

The term “Hypertension”, “high blood pressure” and “HBP” refer to a long-term medical condition in which the blood pressure in the arteries is persistently elevated. Long-term high blood pressure is a major risk factor for stroke, coronary artery disease, heart failure, atrial fibrillation, peripheral arterial disease, vision loss, chronic kidney disease, and dementia.

The terms “Alcoholic liver disease” (ALD) and “alcohol-related liver disease” refer to liver manifestations including fatty liver, alcoholic hepatitis, and chronic hepatitis with liver fibrosis or cirrhosis, resulting from alcohol overconsumption.

The terms “Non-alcoholic fatty liver disease” and “NAFLD” refers to an excessive fat build-up in the liver without another clear cause such as alcohol use. There are two types of NAFLD; non-alcoholic fatty liver (NAFL) and non-alcoholic steatohepatitis (NASH), with the latter also including liver inflammation, scarring and fibrosis. When NAFLD progresses into NASH (10-20% of NAFLD cases), it may eventually lead to complications, such as end stage liver failure (cirrhosis) and hepatocellular carcinoma (HCC). NASH is also associated with increased risk for cardiovascular disease.

“Hepatocellular carcinoma” (HCC) is the most common type of primary liver cancer in adults and is currently the most common cause of death in people with cirrhosis. HCC is the third leading cause of cancer-related death worldwide. It occurs in the setting of chronic liver inflammation and is most closely linked to chronic viral hepatitis infection (hepatitis B or C) or exposure to toxins such as alcohol, aflatoxin, or pyrrolizidine alkaloids. Certain diseases, such as hemochromatosis and alpha 1-antitrypsin deficiency, have been shown to increase the risk of developing HCC. With the obesity pandemic, NASH has become a major risk factor for cirrhosis and HCC.

Overweight and obesity are associated with increased risk of 13 types of cancer, referred to herein as “obesity and overweight related cancers”. These cancers account for about 40 percent of all cancers diagnosed in the United States in 2014. Obesity related cancers as used herein include meningioma (cancer in the tissue covering brain and spinal cord), adenocarcinoma of the esophagus, multiple myeloma (cancer of blood cells), kidney cancer, uterine cancer, ovarian cancer, thyroid cancer, breast cancer in post-menopausal women, liver cancer, gallbladder cancer, cancer in the upper stomach, pancreatic cancer, colon and rectal cancer. Each possibility is a separate embodiment.

The term “treating” as used herein refers to an approach for obtaining beneficial or desired results, including clinical results. Beneficial or desired clinical results can include, but are not limited to, alleviation or amelioration of one or more symptoms or conditions, diminishment of extent of disease, stabilization of the state of disease, prevention of spread or development of the disease or condition, delay or slowing of disease progression, amelioration or palliation of the disease state, and remission (whether partial or total). “Treating” can also mean prolonging survival of a patient beyond that expected in the absence of treatment. “Treating” can also mean inhibiting the progression of disease, slowing the progression of disease temporarily, although more preferably, it involves halting the progression of the disease permanently.

The following examples are presented in order to more fully illustrate some embodiments of the invention. They should in no way be construed, however, as limiting the broad scope of the invention. One skilled in the art can readily devise many variations and modifications of the principles disclosed herein without departing from the scope of the invention.

### EXAMPLES

### Statistics

For experiments 1-10, data are presented as mean±SE; for experiment 7, n=6-7 each. *P value<0.05 between two genotypes; for experiment 8, *P≤0.05; **P≤0.01; ***P≤0.001; for experiment 9, Asterisks/Hash marks depict statistically significant differences, *P≤0.05; **P≤0.01; ***P≤0.001 between WT and FAT10−/− mice, #P≤0.05; ##P≤0.01; ###P≤0.001 between chow and HFD; for experiment 10, *P≤0.05; **P≤0.01; ***P≤0.001 between apoE−/− and apoE−/− FAT10−/− (DKO) mice.

### Example 1—Reducing Expression of FAT10 in Mouse Hepatocyte Cell Line Using Anti-FAT10 ASO

ASOs targeting FAT10 were designed and selected using in-silico prediction algorithms and applications, PFRED (https://pfred.github.io/). Such applications identify potentially potent sequences, by utilizing prediction and scoring algorithms, along with user pre-defined criteria, off-targets analysis, and known quality parameters (such as GC content).

The length for all sequences was 20-nt, and they matched FAT10 without missense. No more than one off-target was allowed, and only of genes not expressed in the liver. GC content for all sequences is set to 45-60%, and they did not contain specific sequences known to be problematic in ASO design (i.e., GGGG).

Flanking regions were 2′-MOE modified, and all cytosines were methylated. All oligonucleotides were chemically modified with phosphorothioate (PS) in the backbone. All oligonucleotides were chemically modified with phosphorothioate (PS) in the backbone, five 2′-methoxyethyl (2′-MOE)-modified ribonucleotides at each terminus and a central region of ten 2′-deoxynucleotide residues recognized by RNase H1 (5-10-5 ‘gapmer’ structure). While the flanking 2′-MOE ends prevent nuclease cleavage of the ASO, the chimeric gapmer ASO design directs RNase H1 to the central gap where it performs specific mRNA degradation. The highest scored ASOs were first examined in-vitro using a synthetic platform (Psi-check) that enables evaluation of the ASO's gene-silencing efficiency using Luciferase reporter (data not shown).

The amino acid sequence of murine FAT10 is set forth in SEQ ID NO: 55, The nucleotide sequence of murine FAT10 is set forth in SEQ ID NO: 56.

Table 1 below provides the sequence of the various ASOs tested against murine FAT10.

TABLE 1
murine FAT10 ASOs

| NAME | SEQ ID NO | Sequence |
|---|---|---|
| ASO4 | 6 | ACTTTGTCATTCTCAGTGGT |
| ASO5 | 7 | CACTTTGTCATTCTCAGTGG |
| ASO6 | 8 | CTTCACTTTGTCATTCTCAG |
| ASO7 | 9 | TTCTTCACTTTGTCATTCTC |
| ASO8 | 10 | TTTGAGCTTCTCATCACCGC |
| ASO10 | 11 | ATAGCTCAGAGGAAATCGGC |
| ASO11 | 12 | GGTAATAGCTCAGAGGAAAT |
| ASO12 | 13 | GGACTCCACCAGAAACAAGG |
| ASO13 | 14 | CCGTTGCAATTCACAACCTG |

Next, hepatocyte cell line (FL38B) cells were transfected with 100 nM of a candidate ASO using lipofectamine 3000 transfection reagent. 24 h post transfection cells were either treated with 10 ng/ml IFNγ for 6 hours or left untreated. FAT10 expression levels were analyzed using RT-PCR using GAPDH expression as an endogenous control, in order to establish ASO efficiency and FAT10 levels.

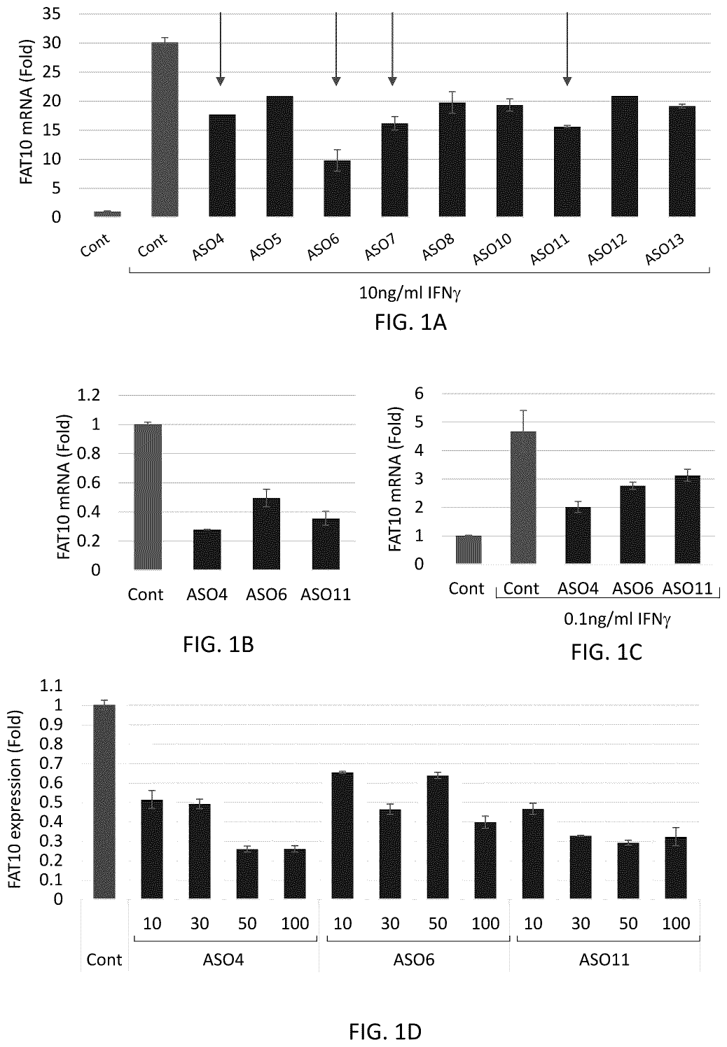

The newly designed ASOs were then analyzed for their ability to reduce FAT10 expression. As seen from FIG. 1A an up to 60% reduction in FAT10 gene transcript levels was obtained.

Three of the ASOs targeting murine FAT10 were further evaluated for their efficiency in inhibiting FAT10 expression in untreated cells (FIG. 1B) and in cells treated with low concentrations of IFNγ (0.1 ng) (FIG. 1C). Advantageously, the ASOs were further shown to provide inhibition of FAT10 expression in a dose dependent manner (FIG. 1D).

### Example 2—Reducing Expression of FAT10 in Primary Mouse Hepatocytes Using FAT10 ASO

Primary mouse hepatocytes were isolated from apoE−/− mice by collagen perfusion and percoll gradient purification as described in Chami-Natan&Goldstein, STAR protocols (2020). The cells were then transfected with 10/30/50/100 nM FAT10-ASO using lipofectamine 3000 transfection reagent. 24 h post transfection, the cells were harvested, RNA was extracted, cDNA synthesized and FAT10 expression analysis performed by RT-PCR using GAPDH expression serving as endogenous control. Data are presented as mean±SE.

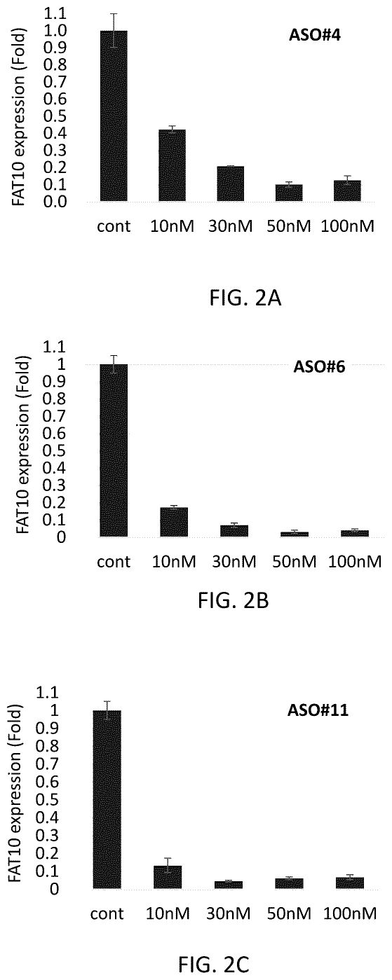

As seen from FIG. 2A-FIG. 2C, the three ASOs tested, namely ASO4, ASO6 and ASO11 showed very efficient (up to 97%) and dose dependent inhibition of FAT10 in the primary murine hepatocytes.

### Example 3—Reducing Expression of FAT10 in Human Hepatocyte Cell Line Using FAT10 ASO

ASOs targeting human FAT10 were designed using in-silico prediction algorithms and modified, as described in Example 1.

Table 2 provides the sequence of the ASOs tested against the human FAT10. Additional sequences of ASOs targeting the human FAT10 are provided in SEQ ID Nos: 15-54

TABLE 2
human FAT10 ASOs

| NAME | SEQ ID NO | Sequence |
|---|---|---|
| ASO260 | 3 | TGGTCCTGCACAGGAACCTT |
| ASO141 | 4 | AGGCTTCTCCGTGGCTTTAA |
| ASO96 | 5 | GGTGCCTCTTTGCCTCATCA |

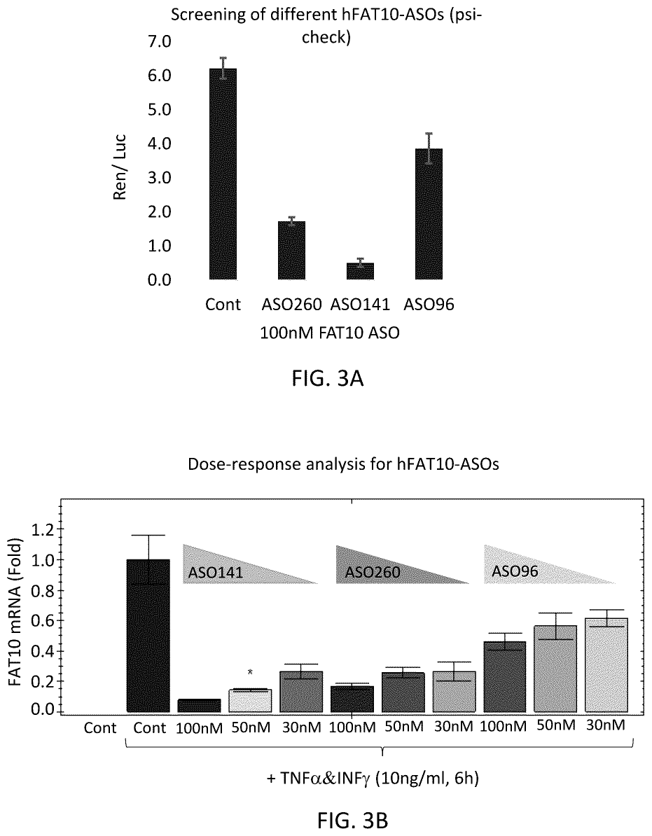

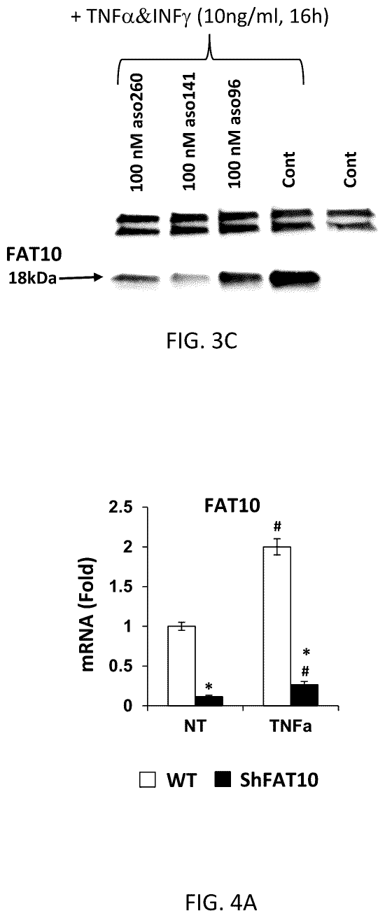

The hFAT10-ASOs were initially screened using a Luciferase reporter-based platform (psi-check). HEK293 cells were transfected with a psi-check plasmid (harboring a human FAT10 sequence and a Luciferase Reporter) and different hFAT10-targeting ASOs (100 nM). Luciferase intensity was measured 48 hours post-transfection. As seen from FIG. 3A, all the tested ASO reduced luciferase levels.

The efficiency of the human ASOs were then tested for their ability to inhibit FAT10 expression in a human hepatocyte cell line (HEPG2). The HEPG2 cells were transfected with 30/50/100 nM FAT10-ASO using lipofectamine 3000. 24-hours post transfection, the cells were stimulated with 10 ng/ml TNF-alpha and INF-gamma (6 h and 16 h for RNA and protein analysis, respectively). RNA and proteins were extracted and analyzed by RT-PCR and western blot, respectively.

Advantageously, as seen from FIG. 3B and FIG. 3C, the tested ASOs caused a dose-dependent reduction in FAT10 transcripts and protein levels, respectively.

### Example 4—FAT10 Knockdown Reduces Expression of SREBP2, SREBP1c and their Target Genes in Mouse Hepatocytes

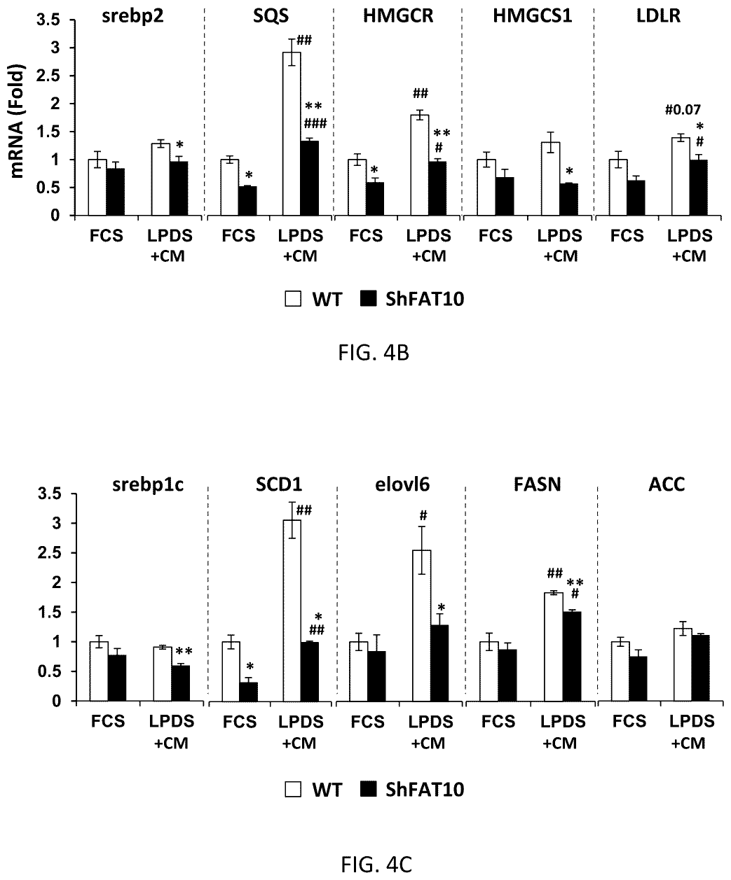

First, a stable FL83B cell line with lentivirus-mediated knockdown of FAT10 (ShFAT10) was generated. FAT10 expression levels were analyzed by RT-PCR using GAPDH expression as an endogenous control. As shown in FIG. 4A, the TNFα-mediated induction of FAT10 was inhibited indicating more than 90% knockdown of FAT10.

In order to activate the SREBP pathway, the medium was changed from 10% fetal calf serum (FCS) to lipoprotein-deficient serum (LPDS) for 24 h. To further activate the SREBP pathway, we added the HMGCR inhibitor Compactin to the medium. Indeed, LPDS increased the expression of SREBP2 and SREBP1c target genes, however this increase was significantly reduced in FAT10 knockdown cells (FIG. 4B and FIG. 4C).

### Example 5—CRISPR Mediated FAT10 Knockdown Reduces Expression of SREBP2 and SREBP2 Target Genes in Human Hepatocytes

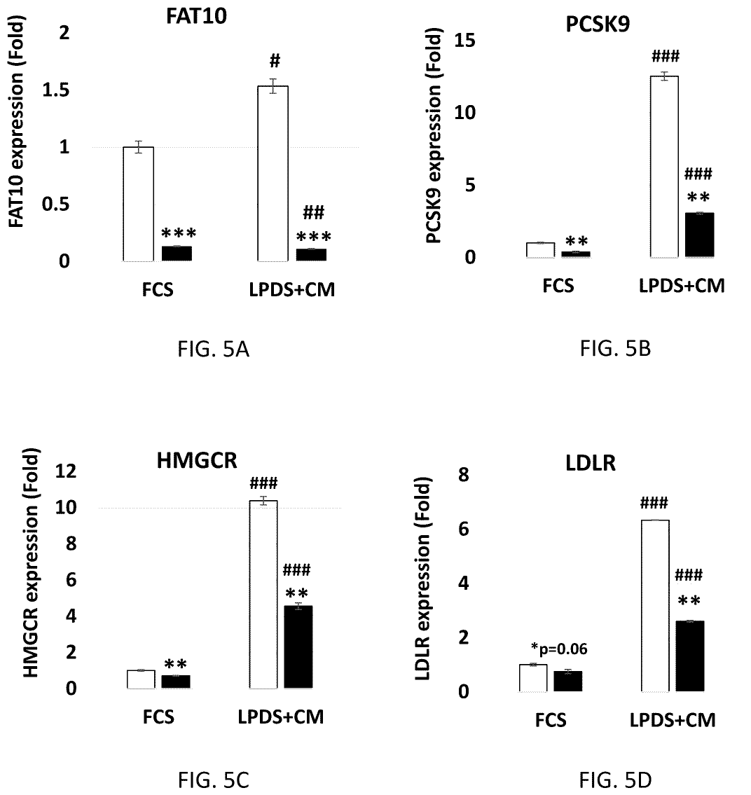

Human hepatocyte cells (HEPG2) were genetically modified using the CRISPR-CAS9 gene editing, as known in the art. FAT10 expression levels were analyzed using RT-PCR using GAPDH expression as an endogenous control to assess the knock-down in cells grown in fetal craft serum supplied growth medium (FCS) or in lipoprotein-deficient serum and conditional growth medium (LPDS+CM) for 24 h. As seen from FIG. 5A, in the control cells, FAT10 expression was high and increased in normal and depleted growth conditions, respectively. As a result of the CRISPR-mediated KD, FAT10 levels reached only residual levels under both growth conditions (black columns—HEPG2-FAT10 CRISPR).

Subsequently, the expression levels of SREBP2 target genes was evaluated by RT-PCR in both growth conditions. As seen from FIG. 5B—FIG. 5D, whereas control cells grown in LPDS+CM had an increased expression level of PCSK9, HMGCR and LDLR, as compared to cells grown under normal growth conditions, the increase was significantly attenuated in the CRISPR-mediated FAT10 knockdown cells.

### Example 6—Injection of GalNac-Conjugated FAT10 ASO to apoE−/− Mice is Safe and Reduces Abdominal Fat Mass and Plasma Cholesterol Levels

In order to assess the effect of injection of GalNac-conjugated FAT10 ASO, two different GalNac-conjugated FAT10ASOs, namely ASO4 (SEQ ID NO: 6) and ASO11 (SEQ ID NO: 12), were injected (10 mg/kg) with three, weekly s.c. injections into apoE−/− hypercholosterolemic mice. The mice were sacrificed 21 days after the last injection and their plasma, livers and epididymal white adipose tissue and harvested for evaluation

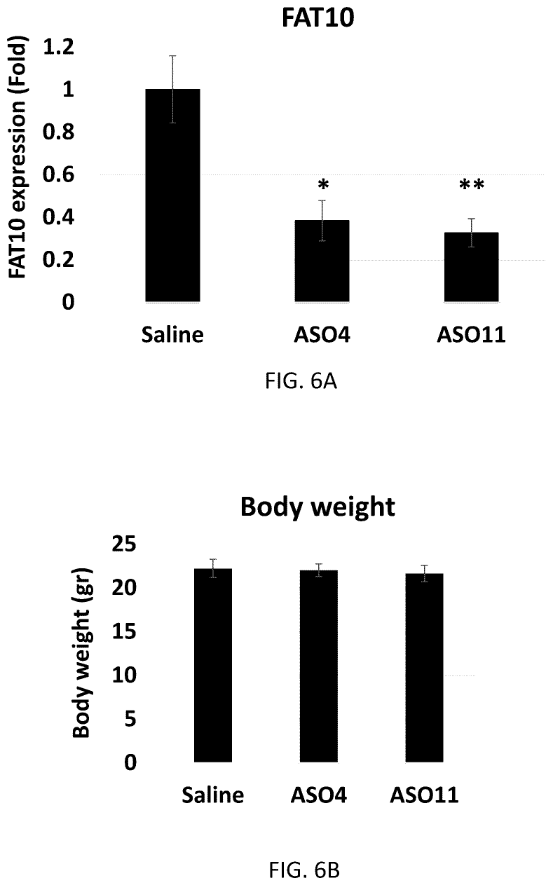

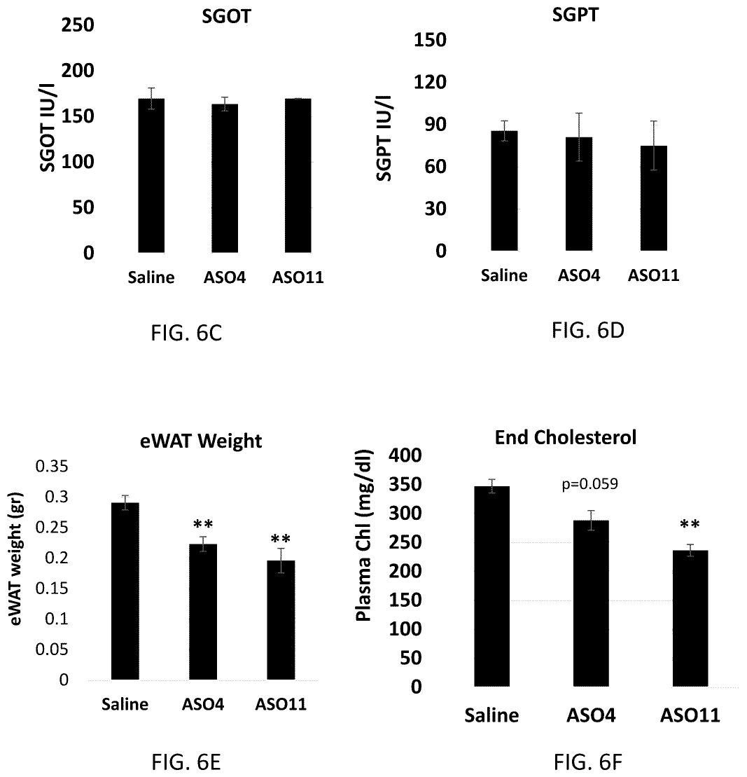

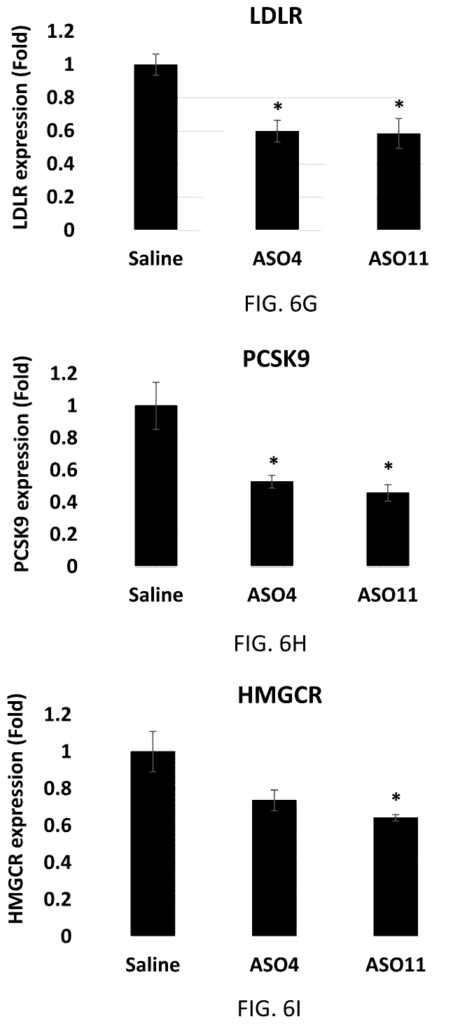

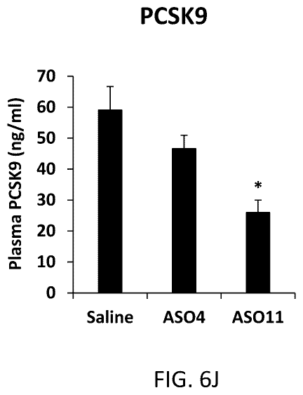

As seen from FIG. 6A, injection of both ASOs resulted in a significant decrease in FAT10 levels (60-70%) in the livers harvested from mice administered with each of the ASOs, as compared to the expression level in control mice, thereby confirming that the FAT10 ASOs are efficiently delivered to the liver where they exert their function.

Advantageously, the overall body weight of the FAT10-ASO administered mice (FIG. 6B), as well as liver function, as evaluated by the SGOT (FIG. 6C) and SGPT (FIG. 6D) liver function tests, was essentially unaltered, indicating the injection of FAT10-ASOs is safe.

Notably, a significant reduction in epididymal white adipose tissue weight (eWAT) was observed as a result of the FAT10 ASO injections already 3 weeks after injection, as seen from FIG. 6E, thus advantageously demonstrating that injection of GalNac-conjugated FAT10-ASOs may contribute to a reduction in abdominal fat mass. This result indicates that hepatic silencing of FAT10 with GalNac-conjugated FAT10-ASOs could be a novel and efficient strategy for treating obesity and its associated-co-morbidities.

Moreover, as seen from FIG. 6F, a marked decline in the total plasma cholesterol levels was observed as a result of the FAT10 ASO injection.

Once again, the reduction in the cholesterol levels concurrent with a reduction in SREBP2 target genes, LDLR, PCSK9, and HMGCR, as seen from FIG. 6G to FIG. 6I. The reduction in PCSK9 expression was also observed in its plasma protein concentrations (FIG. 6J)

### Example 7—Gene Deletion of FAT10 Inhibits Aging-Related Hepatic Accumulation of Triglycerides (TG) and Cholesterol Esters (CE) in Mice

To further assess the effect of attenuating FAT10 expression in-vivo, FAT10−/− whole body knockout mice were used.

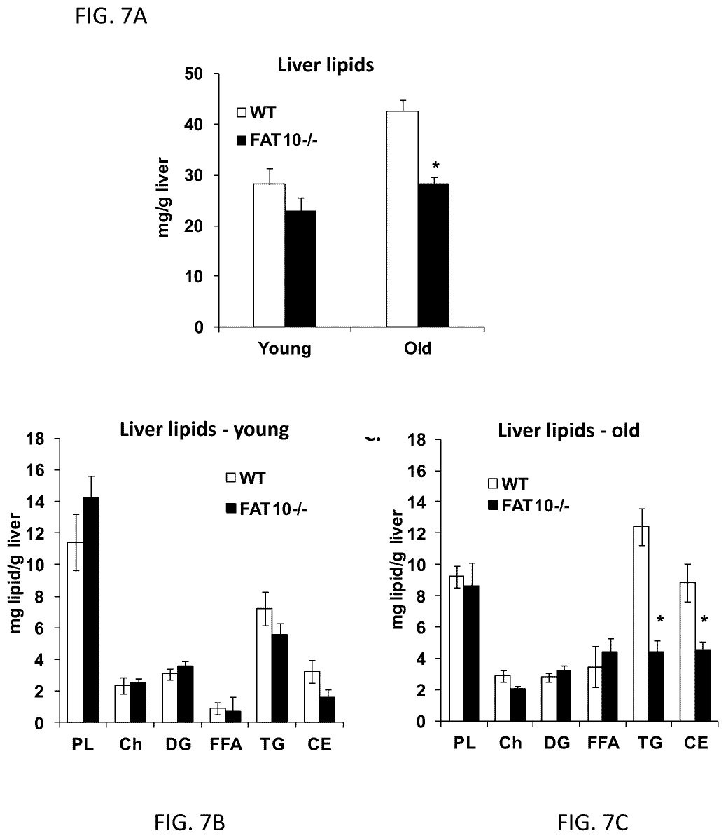

The effect of abolishing FAT10 expression on liver lipids was then assessed in the FAT10 deficient mice (FAT10−/−). Results showed that inactivation of FAT10 inhibits aging-related accumulation of neutral lipids in the liver (FIG. 7A).

Further analysis of the change in total liver lipids, showed that the reduction in hepatic lipid accumulation observed in old FAT10−/− compared with WT mice is due to lower cholesterol ester (CE) and triacylglyceride (TG) (FIG. 7B and FIG. 7C).

### Example 8—Gene Deletion of FAT10 Attenuates Fructose Diet-Induced Activation of SREBP1c and Liver TG Accumulation in Mice

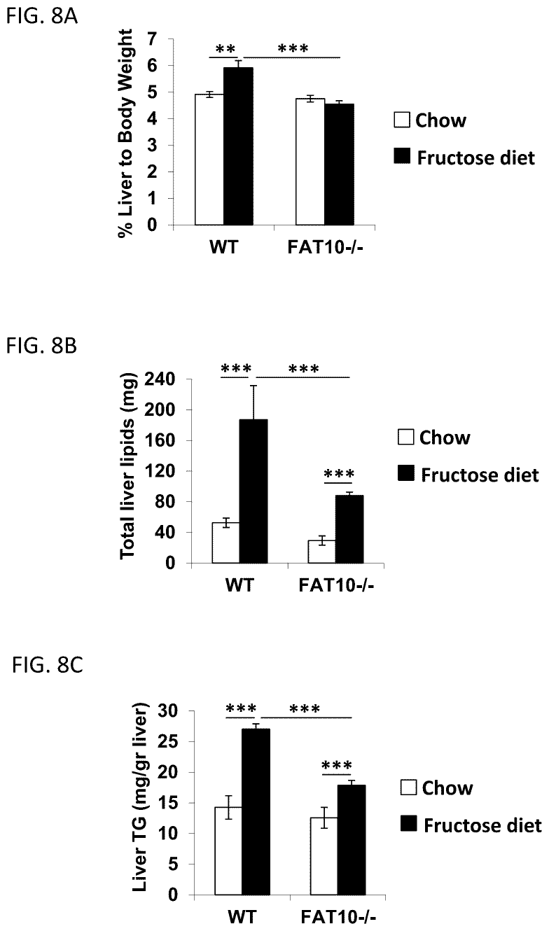

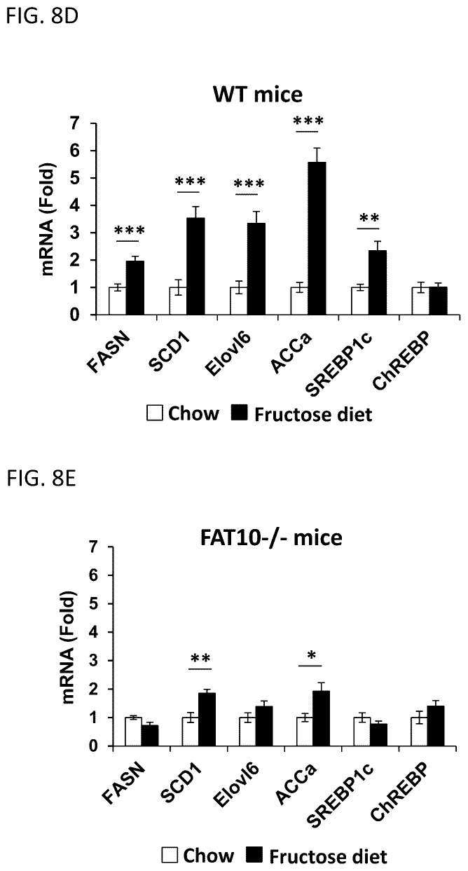

In order to approve that the observed effect of FAT10 deficiency on liver lipids accumulation is an attribute of lipogenesis in hepatocytes, as inferred by the results previously shown in Example 4 (FIGS. 4A-4C), WT and FAT10−/− mice were fed high fructose diet, to induce hepatic lipogenesis and fatty liver (FIG. 8A).

Surprisingly, FAT10 deficiency (FAT10−/−) attenuates the fructose diet-induced liver weight gain (FIG. 8A), total liver lipids (FIG. 8B) and TG accumulation (FIG. 8C), as well as the induction of SREBP1c and its target genes FASN, SCD1, ELOVL6, and ACCa (FIG. 8D and FIG. 8E).

Advantageously, these results indicate that FAT10 deficiency (FAT10−/−) prevents hepatocytes from accumulating fatty acids in the form of triglycerides in the liver, thereby FAT10−/− are at lower risk of developing fatty liver.

### Example 9—Gene Deletion of FAT10 Inhibits the Development of HFD-Induced Obesity and Lowers Plasma Cholesterol, apoB and PCSK9 Levels, as Well as Inhibits Hepatic Activation of SREBP2 and SREBP1c

The effect of abolishing FAT10 expression was also assessed in mice fed high fat diet (HFD) to induce obesity.

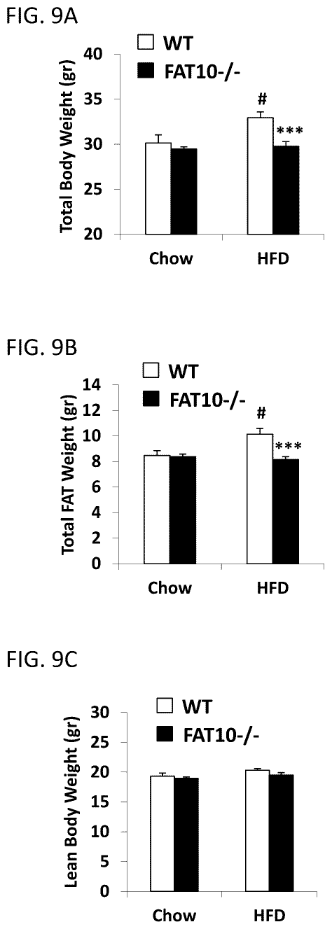

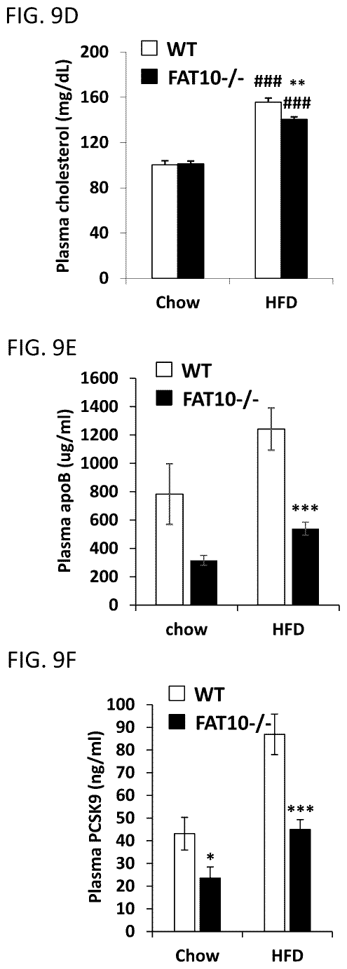

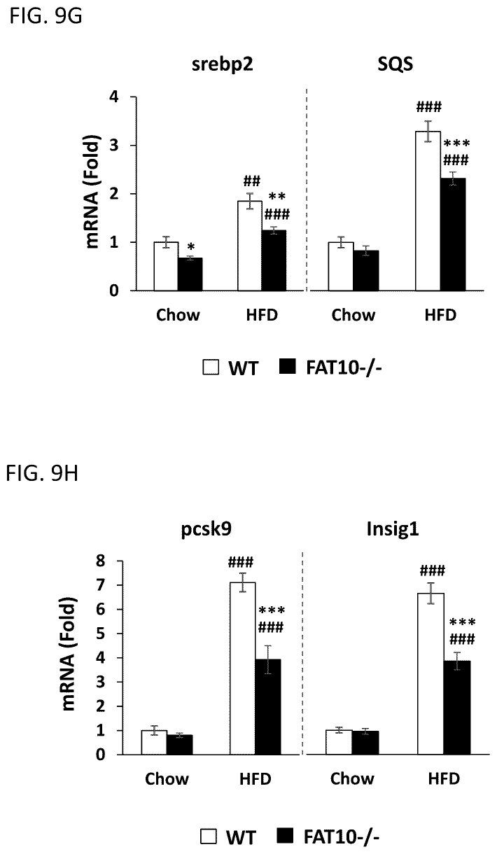

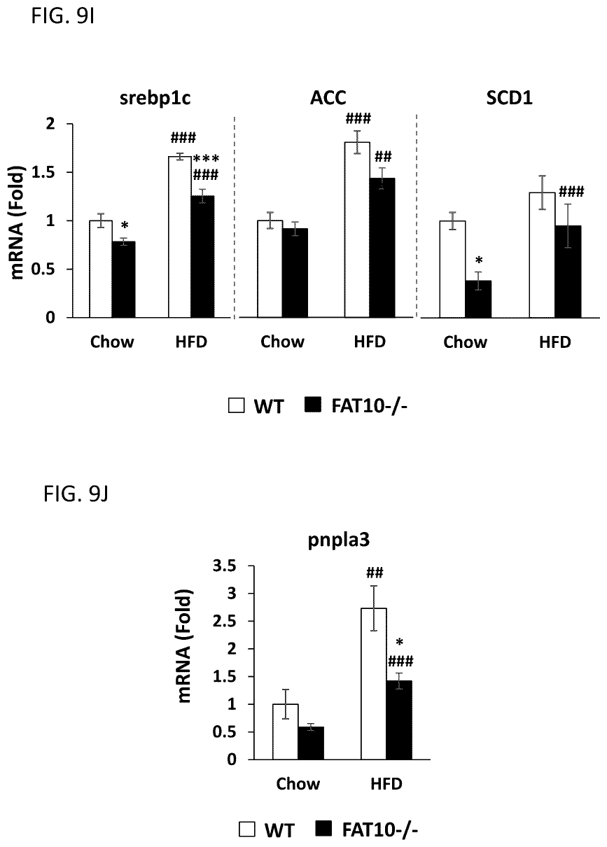

Advantageously, the results showed that FAT10 deficiency inhibits high fat diet (HFD)-induced body weight gain (FIG. 9A) and total body fat (FIG. 9B) with no change in lean body weight (FIG. 9C), indicating that in diet induced obesity, FAT10−/− mice do not gain body fat as WT mice do.

Notably, FAT10 deficiency lowered HFD-induced plasma levels of cholesterol (FIG. 9D), apoB (FIG. 9E) and PCSK9 (FIG. 9F).

Moreover, FAT10 deficiency attenuated HFD-induced hepatic activation of SREBP2, SREBP1c and their target genes SQS, PCSK9, INSIG1, ACC, SCD1, and PNPLA3 in vivo (FIG. 9G-FIG. 9J).

Advantageously, these results indicate that FAT10 deficiency (FAT10−/−) are at lower risk for diet induced obesity.

### Example 10—Gene Deletion of FAT10 Inactivates Hepatic SREBP2, Lowers Plasma VLDL, IDL and LDL Cholesterol and Atherosclerotic Lesion Area in apoE−/− Mice

Next, the effect of FAT10 deficiency (FAT10−/−) was assessed in an in vivo model for atherosclerosis in apoE-deficient mice (apoE−/−). ApoE−/− mice develop hypercholesterolemia, with an increase in cholesterol-rich apoB-containing lipoproteins that constitutes a major risk for development of atherosclerosis.

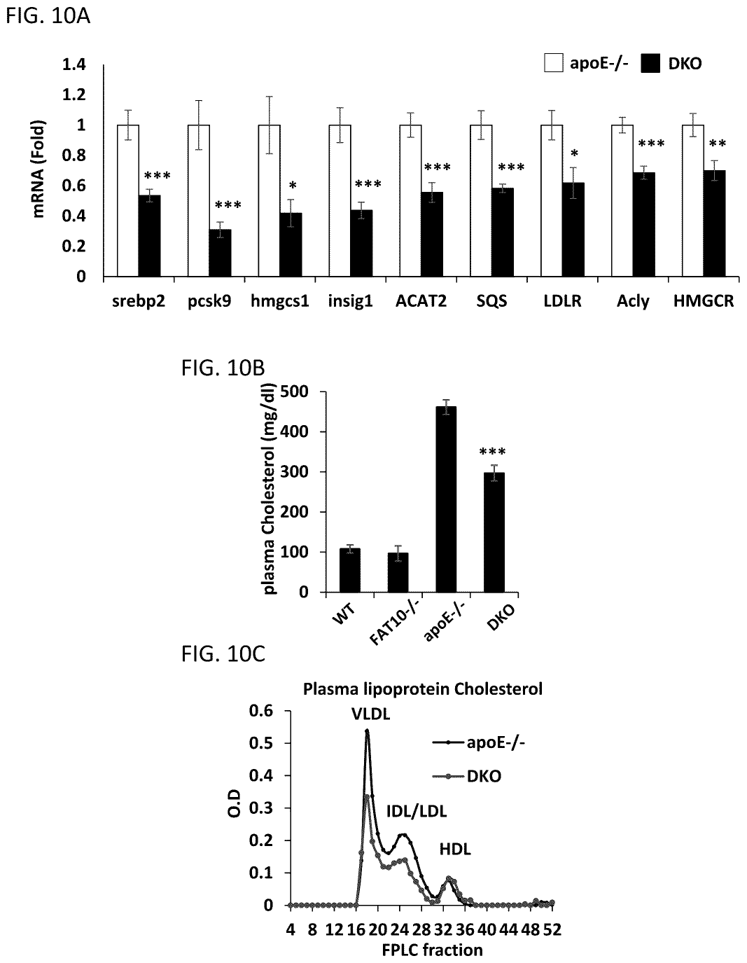

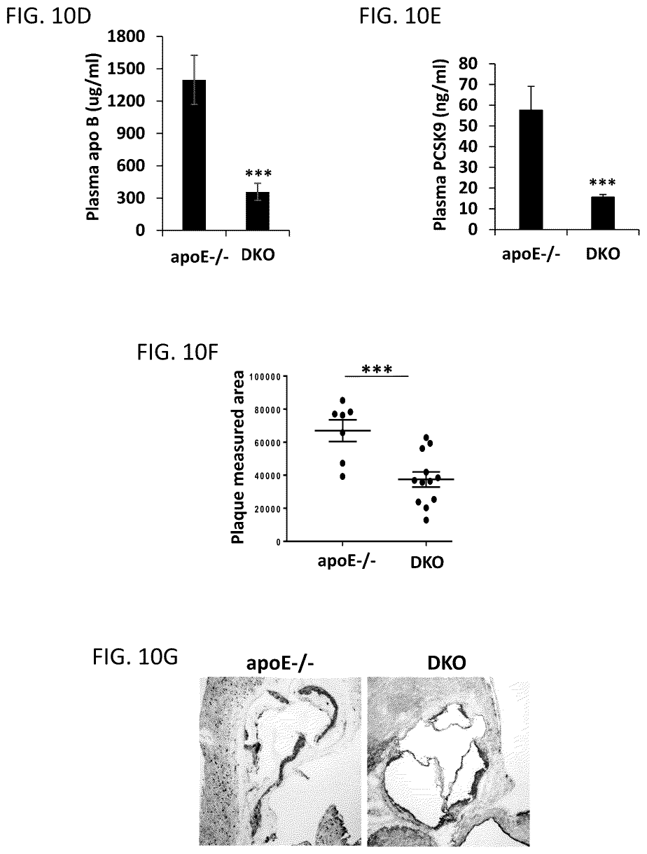

With respect to apoE−/− mice, in apoE−/− FAT10−/− double knockout (DKO) mice gene deletion of FAT10 attenuated hepatic expression of SREBP2 and activation of its target genes PCSK9, HMGCS1, INSIG1, ACAT2, SQS, LDLR, ACLY, and HMGCR (FIG. 10A).

Advantageously, DKO mice showed reduced levels of total plasma cholesterol (FIG. 10B) and of cholesterol carried by apoB-containing lipoproteins VLDL, IDL and LDL, but not HDL (FIG. 10C), thereby indicating that DKO mice are at lower risk for the development of hypercholesterolemia.

Additionally, plasma apoB levels (FIG. 10D), and plasma PCSK9 levels (FIG. 10E) are lowered in DKO compared with apoE−/− mice.

Advantageously, DKO mice showed a reduction in aortic sinus atherosclerotic plaque area, indicating a lowering effect of atherosclerosis development (FIG. 10F and FIG. 10G).

### Example 11—Silencing FAT10 in the Liver of LDLR−/− Mice

Next, to better understand if the observed reduction in levels of plasma cholesterol and cholesterol carried by apoB-containing lipoproteins LDL (FIG. 10B and FIG. 10C), is due to increased clearance into the liver through the LDL receptor (LDLR), experiments are performed to abolish FAT10 expression in LDLR-deficient mice.

In these experiments, LDLR-deficient mice are used as a model of homozygote familial hypercholesterolemia (FH). Three-month-old male LDLR−/− mice fed regular chow diet, are subcutaneously injected weekly with GalNAc-FAT10 ASO, control ASO or saline (n=10 in each group) for 4 weeks. One week after the last injection, mice are killed and body, liver and eWAT weight are measured. In addition, plasma cholesterol, TG and lipoprotein profile (FPLC), and serum ALT are assessed, as well as liver expression of SREBPs and their target genes.

In these experiments if gene silencing of FAT10 will reduce plasma LDL-C in LDLR−/− mice, similarly to the results observed in Example 6 (FIGS. 6F-6G), meaning that FAT10 deficiency lowers plasma cholesterol levels independent of LDLR and indicates that FAT10 neutralization in the liver to lower plasma cholesterol levels could be utilized for treating patients with homozygote FH.

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

1.-19. (canceled)

20. A composition comprising an antisense oligonucleotide (ASO) RNA therapeutic targeting FAT 10 and a suitable carrier, wherein the ASO has at least 90% sequence identity to any of the nucleotide sequences set forth in SEQ ID Nos: 3-5.

21. The composition of claim 20, wherein the ASO comprises any of the nucleotide sequences set forth in SEQ ID Nos: 3-5.

22. The composition of claim 20, wherein the RNA therapeutic is configured to provide liver specific reduction in FAT-10 levels.

23. The composition of claim 20, wherein the RNA therapeutic is conjugated to a GalNac molecule.

24. The composition of claim 20, further comprising a PCSK9 inhibitor.

25. A method for treating familial hypercholesterolemia, dyslipidemia, atherosclerosis, atherosclerotic cardiovascular disease (ASCVD), obesity, type 2 diabetes, hypertension, alcoholic or non-alcoholic fatty liver disease, hepatocellular carcinoma, obesity-associated cancer, or any combination thereof, comprising administering to a subject the composition comprising an antisense oligonucleotide (ASO) RNA therapeutic of claim 20.

26. The method of claim 25, wherein the familial hypercholesterolemia is heterozygotic or homozygotic.

27. The method of claim 25, wherein the ASO comprises a nucleotide sequence set forth in any of SEQ ID Nos: 3-5.

28. The method of claim 25, wherein the RNA therapeutic is configured to provide liver specific reduction in FAT-10 levels.

29. The method of claim 25, wherein the RNA therapeutic is conjugated to N-Acetylgalactosamine (GalNac).

30. The method of claim 25, wherein the treating comprises inhibiting attenuating or preventing accumulation of triglycerides and/or cholesterol.

31. The method of claim 30, wherein the accumulation is hepatic accumulation of triglycerides and/or cholesterol.

32. The method of claim 25, wherein the RNA therapeutic further comprises a PCSK9 inhibitor.
